(19) United States
(12) Patent Application Publication
(10) Pub. No.: US 2020/0170594 A1
(43) Pub. Date: Jun. 4, 2020

(54) MOBILE RADIOGRAPHIC IMAGING APPARATUS, OPERATION METHOD OF MOBILE RADIOGRAPHIC IMAGING APPARATUS, AND OPERATION PROGRAM OF MOBILE RADIOGRAPHIC IMAGING APPARATUS

(71) Applicant: FUJIFILM Corporation, Tokyo (JP)

(72) Inventors: Kazuhiro MAKINO, Kanagawa (JP); Ryo IMAMURA, Kanagawa (JP)

(73) Assignee: FUJIFILM Corporation, Tokyo (JP)

(21) Appl. No.: 16/698,967

(22) Filed: Nov. 28, 2019

(30) Foreign Application Priority Data
Nov. 30, 2018 (JP) 2018-225681

(51) Int. Cl. A61B 6/00

(52) U.S. Cl. CPC A61B 6/4405; A61B 2562/02; A61B 2560/0431; A61B 6/548

(57) ABSTRACT

In the mobile radiographic imaging apparatus, a first detection sensor detects a first operation of an operator on a handle, and a second detection sensor detects a second operation of the operator, which is different from the first operation and is performed within a set range that is set in advance around a carriage unit. A mode controller continues execution of an automatic travel mode in a case where the first operation is not detected by the first detection sensor and the second operation is continuously detected by the second detection sensor. The mode controller stops the automatic travel mode and executes the manual travel mode in a case where the first operation is detected by the first detection sensor during execution of the automatic travel mode.

### CROSS-REFERENCE TO RELATED APPLICATIONS

The present application claims priority under 35 U.S.C. § 119 to Japanese Patent Application No., 2018-225681 filed on Nov. 30, 2018. The above application is hereby expressly incorporated by reference, in its entirety, into the present application.

### BACKGROUND

### 1. Technical Field

The technique of the present disclosure relates to a mobile radiographic imaging apparatus, an operation method of a mobile radiographic imaging apparatus, and an operation program of a mobile radiographic imaging apparatus.

### 2. Description of the Related Art

A mobile radiographic imaging apparatus that performs radiographic imaging while going around a patient room in a hospital is known. The mobile radiographic imaging apparatus comprises a carriage unit and a handle. The carriage unit has a plurality of wheels for traveling. The handle is gripped by an operator, such as a radiology technician, in order to steer the carriage unit. The operator determines the travel speed and travel direction of the carriage unit by operating the handle to change the way in which the force is applied to the handle or to adjust the direction in which the force is applied to the handle.

A mobile radiographic imaging apparatus described in JP2006-141669A comprises an automatic travel mode in which the carriage unit travels without a handle operation of the operator in addition to a manual travel mode in which the carriage unit travels by the handle operation of the operator. In the mobile radiographic imaging apparatus described in JP2006-141669A, in a case where the operator operates a handle during the execution of the automatic travel mode, the automatic travel mode is stopped and the manual travel mode is performed.

### SUMMARY

In the mobile radiographic imaging apparatus described in JP2006-141669A, once the automatic travel mode is started, the automatic travel mode is continued even in a case where there is a distance between the operator and the mobile radiographic imaging apparatus and the operator has moved away to a position where the handle cannot be gripped immediately. From the viewpoint of ensuring higher safety in the automatic travel mode, there is a concern about the state in which the execution of the automatic travel mode is continued even in a case where the operator has moved away to a position where the handle cannot be gripped immediately as described above.

It is an object of the technique of the present disclosure to provide a mobile radiographic imaging apparatus, an operation method of a mobile radiographic imaging apparatus, and an operation program of a mobile radiographic imaging apparatus capable of realizing a safer automatic travel mode.

In order to achieve the aforementioned object, a mobile radiographic imaging apparatus of the present disclosure comprises: a carriage unit which has wheels for traveling and on which a main body unit is mounted; a handle that is provided in the main body unit to steer the carriage unit; a first detection sensor that detects a first operation of an operator on the handle; a second detection sensor that detects a second operation of the operator, which is different from the first operation and is performed within a set range that is set in advance around the carriage unit; and a mode controller that executes or stops an automatic travel mode, in which the carriage unit travels without an operation of the operator on the handle, or a manual travel mode, in which the carriage unit travels by an operation of the operator on the handle, according to a detection result of the first operation and a detection result of the second operation and that continues execution of the automatic travel mode in a case where the first operation is not detected by the first detection sensor and the second operation is continuously detected by the second detection sensor and stops the automatic travel mode and executes the manual travel mode in a case where the first operation is detected by the first detection sensor during execution of the automatic travel mode.

It is preferable that, in a case where the second operation is not detected by the second detection sensor, the mode controller stops the automatic travel mode.

It is preferable that the first detection sensor detects that the operator has gripped the handle as the first operation and the second detection sensor is provided in the main body unit and detects that one hand of the operator has come into contact with the second detection sensor itself as the second operation.

It is preferable that the second detection sensor is attached to one end of an extension cable and is usable by being detached from the main body unit. It is preferable that the second detection sensor has a wireless transmission function for wirelessly transmitting the detection result of the second operation within a communication range limited by the set range.

It is preferable that the first detection sensor is at least one of a sensor that detects contact of a hand of the operator with the handle, a camera that images a gripping state of the handle by the operator, or a sensor that detects that a force for steering the carriage unit has been applied to the handle.

It is preferable that the first detection sensor detects that the operator has gripped the handle with both hands as the first operation and the second detection sensor detects that one hand of the operator has come into contact with the handle as the second operation.

It is preferable that each of the first detection sensor and the second detection sensor is at least one of a sensor that detects contact of a hand of the operator with the handle or a camera that images a gripping state of the handle by the operator.

It is preferable that the second detection sensor has an identification function for identifying the operator. In this case, it is preferable that the mode controller changes a level of the automatic travel mode according to an identification result of the operator by the identification function.

It is preferable that, in a case where a travel environment of the carriage unit does not satisfy recommendation conditions of the automatic travel mode during execution of the automatic travel mode, the mode controller executes manual travel recommendation processing for recommending switching to the manual travel mode.

It is preferable that the manual travel recommendation processing is processing for stopping the automatic travel mode. It is preferable that the manual travel recommendation processing is processing for reducing a travel speed of the carriage unit. It is preferable that the manual travel recommendation processing is processing for providing notification that recommends switching to the manual travel mode.

It is preferable that a parking position of the carriage unit in the automatic travel mode is a position corresponding to a schedule of radiographic imaging, and includes a position outside a patient room that is defined in advance for each patient room.

An operation method of a mobile radiographic imaging apparatus of the present disclosure is an operation method of a mobile radiographic imaging apparatus comprising a carriage unit which has wheels for traveling and on which a main body unit is mounted and a handle that is provided in the main body unit to steer the carriage unit. The method comprises: a first acquisition step of acquiring a detection result of a first operation of an operator on the handle from a first detection sensor; a second acquisition step of acquiring a detection result of a second operation of the operator, which is different from the first operation and is performed within a set range that is set in advance around the carriage unit, from a second detection sensor; and a mode control step of executing or stopping an automatic travel mode, in which the carriage unit travels without an operation of the operator on the handle, or a manual travel mode, in which the carriage unit travels by an operation of the operator on the handle, according to a detection result of the first operation and a detection result of the second operation and of continuing execution of the automatic travel mode in a case where the first operation is not detected by the first detection sensor and the second operation is continuously detected by the second detection sensor and stopping the automatic travel mode and executing the manual travel mode in a case where the first operation is detected by the first detection sensor during execution of the automatic travel mode.

An operation program of a mobile radiographic imaging apparatus of the present disclosure is an operation program of a mobile radiographic imaging apparatus comprising a carriage unit which has wheels for traveling and on which a main body unit is mounted and a handle that is provided in the main body unit to steer the carriage unit. The operation program causes a computer to function as: a first acquisition unit that acquires a detection result of a first operation of an operator on the handle from a first detection sensor; a second acquisition unit that acquires a detection result of a second operation of the operator, which is different from the first operation and is performed within a set range that is set in advance around the carriage unit, from a second detection sensor; and a mode controller that executes or stops an automatic travel mode, in which the carriage unit travels without an operation of the operator on the handle, or a manual travel mode, in which the carriage unit travels by an operation of the operator on the handle, according to a detection result of the first operation and a detection result of the second operation and that continues execution of the automatic travel mode in a case where the first operation is not detected by the first detection sensor and the second operation is continuously detected by the second detection sensor and stops the automatic travel mode and executes the manual travel mode in a case where the first operation is detected by the first detection sensor during execution of the automatic travel mode.

According to the technique of the present disclosure, it is possible to provide a mobile radiographic imaging apparatus, an operation method of a mobile radiographic imaging apparatus, and an operation program of a mobile radiographic imaging apparatus capable of realizing a safer automatic travel mode.

### BRIEF DESCRIPTION OF THE DRAWINGS

Exemplary embodiments according to the technique of the present disclosure will be described in detail based on the following figures, wherein:

FIG. 1 is a diagram showing a mobile radiographic imaging apparatus.

FIG. 2 is a diagram showing the rotation direction and the movement direction of a column unit, the movement direction of an arm unit, and the rotation direction of an irradiation unit.

FIG. 3 is a diagram showing the rotation direction of the irradiation unit;

FIG. 4 is a diagram showing the relationship between a carriage unit and a set range;

FIG. 5 is a diagram showing a main body unit viewed from the upper surface side;

FIG. 6 is a block diagram of the mobile radiographic imaging apparatus;

FIG. 7 is a block diagram showing a controller of a CPU of the mobile radiographic imaging apparatus;

FIG. 8 is a block diagram showing a carriage unit controller;

FIG. 9 is a diagram showing an example of map information;

FIG. 10 is a diagram showing an example of imaging schedule information;

FIG. 11 is a diagram showing a route along which the carriage unit travels in a case where imaging schedule information is the content shown in FIG. 10;

FIG. 12 is a timing chart showing the detection and non-detection of a first operation, the detection and non-detection of a second operation, and the state of the carriage unit;

FIG. 13 is a timing chart showing the detection and non-detection of the first operation, the detection and non-detection of the second operation, and the state of the carriage unit;

FIG. 14 is a timing chart showing the detection and non-detection of the first operation, the detection and non-detection of the second operation, and the state of the carriage unit;

FIG. 15 is a table summarizing the content of FIGS. 12 to 14;

FIG. 16 is a diagram showing a state in which an automatic travel mode is being executed;

FIG. 17 is a diagram showing a state in which the automatic travel mode is stopped and a manual travel mode is executed;

FIG. 18 is a flowchart showing the procedure of automatic travel mode control processing;

FIG. 19 is a diagram showing an example in which a second detection sensor having a wireless transmission function is used;

FIG. 20 is a diagram showing a state in which the automatic travel mode is being executed in an example in which a first detection sensor configured by a camera is used;

FIG. 21 is a diagram showing a state in which the automatic travel mode is stopped and the manual travel mode is executed in an example in which a first detection sensor configured by a camera is used;

FIG. 22 is a diagram showing a state in which the automatic travel mode is being executed in an example in which a first detection sensor configured by a pressure sensor is used;

FIG. 23 is a diagram showing a state in which the automatic travel mode is stopped and the manual travel mode is executed in an example in which a first detection sensor configured by a pressure sensor is used;

FIG. 24 is a diagram showing a second embodiment in which it is detected as a second operation that one hand of an operator has come into contact with a handle;

FIG. 25 is a diagram showing a state in which the automatic travel mode is being executed in the second embodiment;

FIG. 26 is a diagram showing the second embodiment in which it is detected as a first operation that the operator has gripped a handle with both hands;

FIG. 27 is a diagram showing a state in which the automatic travel mode is stopped and the manual travel mode is executed in the second embodiment;

FIG. 28 is a table summarizing the state of the carriage unit for detection and non-detection of the first operation and detection and non-detection of the second operation in the second embodiment;

FIG. 29 is a diagram showing a state in which the automatic travel mode is being executed in an example in which a first and second detection sensor configured by a camera is used;

FIG. 30 is a diagram showing a state in which the automatic travel mode is stopped and the manual travel mode is executed in an example in which a first and second detection sensor configured by a camera is used;

FIG. 31 is a diagram showing a third embodiment in which the level of the automatic travel mode is changed according to the identification result of an operator by the identification function of the second detection sensor;

FIG. 32 is a block diagram showing a carriage unit controller of a fourth embodiment in which manual travel recommendation processing for recommending switching to the manual travel mode is executed;

FIG. 33 is a diagram showing manual travel recommendation processing for stopping the automatic travel mode;

FIG. 34 is a diagram showing manual travel recommendation processing for reducing the travel speed of a carriage unit;

FIG. 35 is a diagram showing manual travel recommendation processing for providing notification that recommends switching to the manual travel mode;

FIG. 36 is a diagram showing a warning screen;

FIG. 37 is a diagram showing map information in which a prohibited area of the automatic travel mode is set; and

FIG. 38 is a diagram showing how the manual travel recommendation processing is executed in a case where the carriage unit moves into the prohibited area of the automatic travel mode.

### DETAILED DESCRIPTION

### First Embodiment

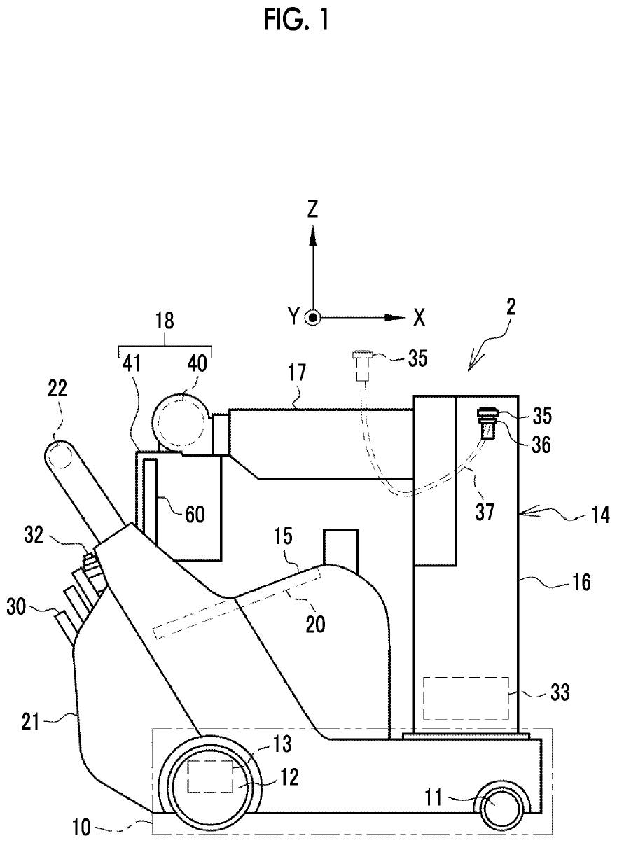

In FIG. 1, a mobile radiographic imaging apparatus 2 comprises a carriage unit 10. The carriage unit 10 has a front wheel 11, a rear wheel 12, and a rear wheel driving unit 13. The front wheel 11 and the rear wheel 12 are examples of “wheels” according to the technique of the present disclosure.

The front wheel 11 is a pair of left and right casters that revolve around the Z axis indicating the height direction of the mobile radiographic imaging apparatus 2. The rear wheel 12 is also a pair of left and right like the front wheel 11, but does not revolve around the Z axis. However, the rear wheel 12 is rotated around the Y axis indicating the width direction of the mobile radiographic imaging apparatus 2 by the rear wheel driving unit 13. The front wheel 11 rotates following the rotation of the rear wheel 12. That is, the carriage unit 10 has four wheels and is of a rear wheel drive type. The carriage unit 10 is not limited to the rear wheel drive type, and may be of a front wheel drive type in which the front wheel 11 rotates around the Y axis by a front wheel driving unit. Alternatively, the carriage unit 10 may be an all-wheel drive type in which the front wheel 11 rotates around the Y axis by the front wheel driving unit and the rear wheel 12 rotates around the Y axis by the rear wheel driving unit.

The rear wheel driving unit 13 is two motors connected to the left and right rear wheels 12. The rear wheel driving unit 13 rotates the left and right rear wheels 12 independently of each other. For this reason, in a case where the rotation speed of the right rear wheel 12 is made faster than that of the left rear wheel 12 by the rear wheel driving unit 13, the mobile radiographic imaging apparatus 2 turns to the left. On the other hand, in a case where the rotation speed of the left rear wheel 12 is made faster than that of the right rear wheel 12 by the rear wheel driving unit 13, the mobile radiographic imaging apparatus 2 turns to the right.

The mobile radiographic imaging apparatus 2 can be moved in the hospital by the carriage unit 10. The mobile radiographic imaging apparatus 2 is used for so-called round-visit imaging in which radiographic imaging is performed while going around a patient room. For this reason, the mobile radiographic imaging apparatus 2 is also called a round-visit car. In addition, the mobile radiographic imaging apparatus 2 can also be brought into the operating room to perform radiographic imaging in the midst of surgery.

A main body unit 14 is mounted on the carriage unit 10. The main body unit 14 includes a central unit 15, a column unit 16, an arm unit 17, an irradiation unit 18, and the like. The mobile radiographic imaging apparatus 2 is moved in a state shown in FIG. 1 in which the irradiation unit 18 is housed in the upper portion of the central unit 15.

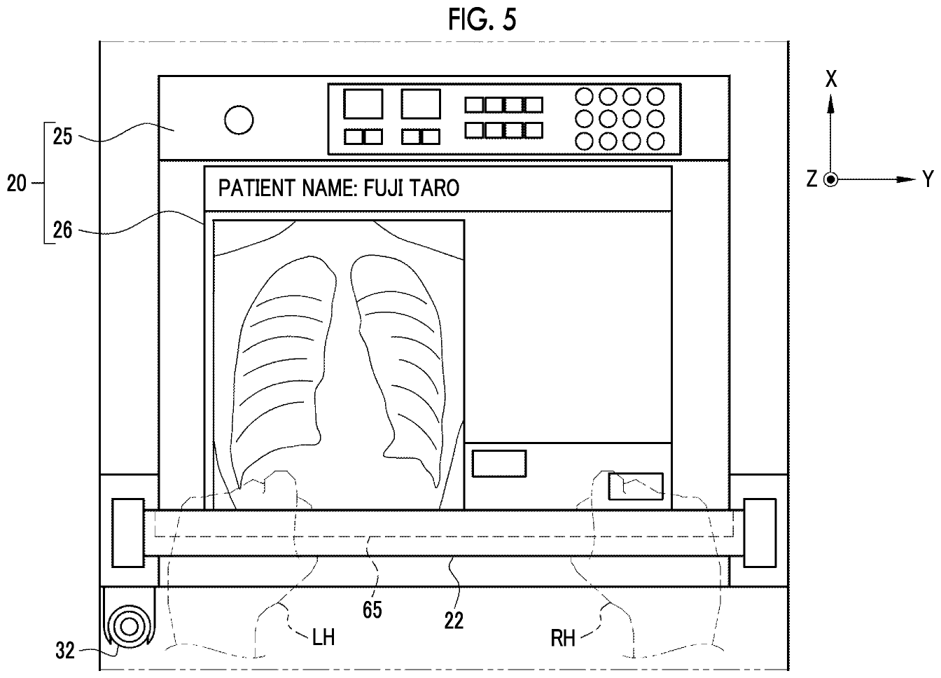

The central unit 15 has a console 20, a cassette housing unit 21, and a handle 22. The console 20 is embedded on the inclined upper surface of the central unit 15. The console 20 is configured to include an operation console 25 and a display 26 (refer to FIG. 5). The operation console 25 is operated by an operator OP at the time of setting the irradiation conditions, at the time of selecting whether or not to execute an automatic travel mode AM to be described later, and the like. The display 26 displays various screens including an irradiation conditions setting screen, a radiographic image, and the like. The selection of whether or not to execute the automatic travel mode AM is performed by displaying a screen asking the operator OP whether or not to execute the automatic travel mode AM on the display 26 immediately after the mobile radiographic imaging apparatus 2 is turned on, for example.

The cassette housing unit 21 is disposed on the back surface of the central unit 15. An electronic cassette 30 is housed in the cassette housing unit 21. As is well known, the electronic cassette 30 is a radiographic image detector that detects a radiographic image expressed by an electrical signal based on radiation transmitted through a subject, and is a portable radiographic image detector capable of performing wireless communication using a built-in battery. There are a plurality of types of electronic cassettes 30 having vertical and horizontal sizes of 17 inches×17 inches, 17 inches×14 inches, 12 inches×10 inches, and the like. In the cassette housing unit 21, a plurality of types of electronic cassettes 30 can be housed regardless of the type. The cassette housing unit 21 has a function of charging the battery of the housed electronic cassette 30.

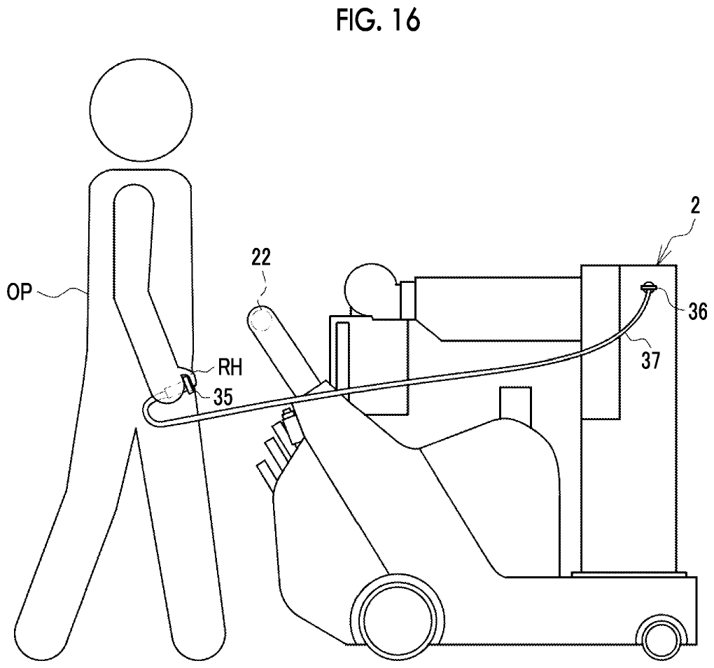

The handle 22 is provided at a position protruding above the central unit 15. The handle 22 has a cylindrical shape that is long in the Y-axis direction (refer to FIG. 5). The handle 22 is gripped by the operator OP (refer to FIG. 16 and the like), such as a radiology technician, in order to steer the carriage unit 10.

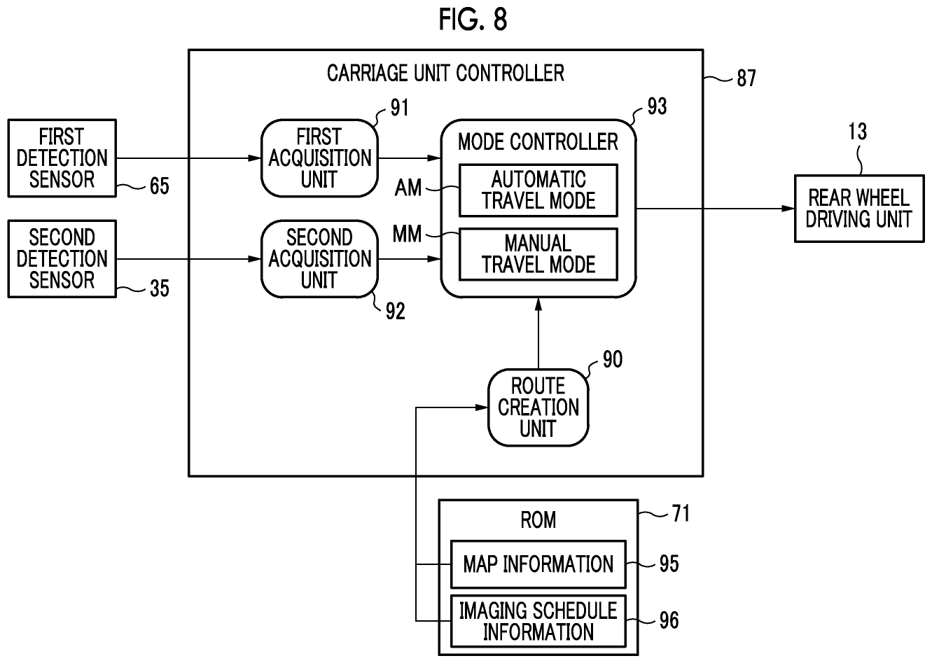

The mobile radiographic imaging apparatus 2 comprises the automatic travel mode AM and a manual travel mode MM (refer to FIG. 8 for both). The automatic travel mode AM is a mode in which the carriage unit 10 travels without an operation of the operator OP on the handle 22. On the other hand, the manual travel mode MM is a mode in which the carriage unit 10 travels by the operation of the operator OP on the handle 22.

Here, the operation of the operator OP on the handle 22 is an operation in which the operator OP grips the handle 22 to change the way in which the force is applied to the handle 22 or adjust the direction in which the force is applied to the handle 22. In the automatic travel mode AM, the carriage unit 10 travels only by the rotation of the rear wheel 12 by the rear wheel driving unit 13 without such an operation on the handle 22.

On the other hand, in the manual travel mode MM, by the operation on the handle 22 described above, the operator OP independently determines the travel speed and the travel direction of the carriage unit 10 to cause the carriage unit 10 to travel. Also in the manual travel mode MM, the rear wheel driving unit 13 is driven. However, the driving of the rear wheel driving unit 13 in the manual travel mode MM is a driving according to the force applied to the carriage unit 10 by the operator OP. For this reason, in the manual travel mode MM, the carriage unit 10 naturally does not travel unless the force of the operator OP is applied. On the contrary, in the manual travel mode MM, the carriage unit 10 does not travel only with the force of the operator OP, and the carriage unit 10 travels only with the assistance of the rear wheel driving unit 13. The force applied to the carriage unit 10 by the operator OP is detected using, for example, a piezoelectric sensor, and the rear wheel driving unit 13 is driven according to the detection result.

The automatic travel mode AM can be realized by using a technique of a conventionally known automated guided vehicle (AGV). Specifically, first, the current position of the carriage unit 10 in the hospital is detected. Then, a distance and a direction from the detected current position to the target position are calculated, and the rear wheel driving unit 13 is driven according to the calculated distance and direction.

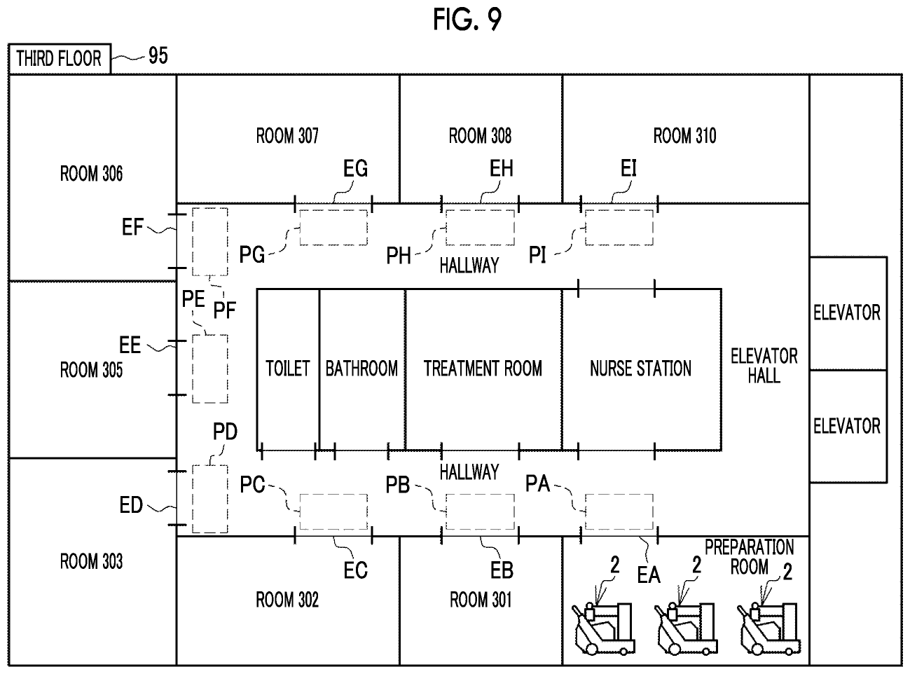

As a method of detecting the current position of the carriage unit 10 in the hospital, for example, the following method can be considered. That is, sensors that detect the travel distance and the travel direction of the carriage unit 10, such as a rotary encoder that detects the rotation amount of the rear wheel 12, a gyro sensor, and an acceleration sensor, are provided in the mobile radiographic imaging apparatus 2. Then, the current position of the carriage unit 10 in the hospital is detected by comparing the travel distance and the travel direction detected by such sensors with map information 95 (FIGS. 8 and 9).

In addition, the following method may be adopted. That is, markers that are different for each position are disposed at a plurality of positions in the hospital. A camera is mounted in the mobile radiographic imaging apparatus 2, and each marker is imaged by the camera so that the marker is image-recognized. Then, the position indicated by the image-recognized marker is detected as the current position of the carriage unit 10 in the hospital. In addition to the methods described above, a method in which a magnetic material is embedded in the hallway and a magnetic field generated by the magnetic material is detected by a magnetic sensor or a method using a distance ranging sensor, such as laser imaging detection and ranging (LIDAR), may be adopted.

An irradiation switch 32 is attached to the upper portion of the cassette housing unit 21. The irradiation switch 32 is a switch for the operator OP to give an instruction to start the emission of radiation. An extension cable (not shown) is connected to the irradiation switch 32, so that the irradiation switch 32 can be used by being detached from the central unit 15. The irradiation switch 32 is, for example, a two-stage pressing type switch. The irradiation switch 32 generates a warm-up command signal in a case where the irradiation switch 32 is pressed to the first stage (half-pressed), and generates an irradiation start command signal in a case where the irradiation switch 32 is pressed to the second stage (fully pressed). Although not shown, a battery for supplying power to each unit is built into the central unit 15.

The column unit 16 has a prismatic shape, and is erected along the Z-axis direction. The column unit 16 is disposed at a position above the front wheel 11 and at the center of the carriage unit 10 in the Y-axis direction. A voltage generator 33 is provided in the column unit 16.

In the upper portion of the column unit 16, a second detection sensor 35 is attached by a fixture 36. The second detection sensor 35 detects a second operation of the operator OP that is different from a first operation. The second operation is a continuous operation of the operator OP for continuing the execution of the automatic travel mode AM. In addition, the second operation is an operation performed within a set range that is set in advance around the carriage unit 10.

The second detection sensor 35 detects that one hand of the operator OP has come into contact with the second detection sensor 35 itself as the second operation. The second detection sensor 35 is, for example, a sensor that detects a hand contact based on the capacitance change or a sensor that detects a hand contact based on the temperature change.

As indicated by a broken line, the second detection sensor 35 is attached to one end of an extension cable 37. With this extension cable 37, the second detection sensor 35 can be used by being detached from the fixture 36.

The arm unit 17 has a prismatic shape similarly to the column unit 16. The arm unit 17 has a proximal end attached to the column unit 16 and a distal end, which is a free end opposite to the proximal end and to which the irradiation unit 18 is attached.

The irradiation unit 18 is configured to include a radiation tube 40 and an irradiation field limiter 41. The radiation tube 40 generates, for example, X-rays as radiation. A filament, a target, a grid electrode, and the like (all not shown) are provided in the radiation tube 40. A voltage from the voltage generator 33 is applied between the filament serving as a cathode and the target serving as an anode. A voltage applied between the filament and the target is called a tube voltage. The filament emits thermoelectrons according to the applied tube voltage toward the target. The target emits radiation by the impact of thermoelectrons from the filament. The grid electrode is disposed between the filament and the target. The grid electrode changes the flow rate of thermoelectrons from the filament toward the target in accordance with the voltage applied from the voltage generator 33. The flow rate of thermoelectrons from the filament toward the target is called a tube current. The tube voltage and the tube current are set as irradiation conditions together with the irradiation time.

In a case where the irradiation switch 32 is half-pressed to generate a warm-up command signal, the filament is preheated and simultaneously the rotation of the target is started. Warm-up is completed at a point in time at which the filament reaches a specified temperature and the target reaches a specified speed. In a state in which the warm-up is completed, in a case where the irradiation switch 32 is fully pressed to generate an irradiation start command signal, a tube voltage is applied from the voltage generator 33 and radiation is generated from the radiation tube 40. In a case where the irradiation time set in the irradiation conditions has passed from the start of the generation of radiation, the application of the tube voltage is stopped and the emission of the radiation is ended.

The irradiation field limiter 41 limits the irradiation field of the radiation generated from the radiation tube 40. The irradiation field limiter 41 has, for example, a configuration in which four shielding plates, such as lead for shielding radiation, are disposed on the sides of a quadrangle and a quadrangular exit opening that transmits radiation is formed in the central portion. The irradiation field limiter 41 changes the size of the exit opening by changing the position of each shielding plate, thereby changing the irradiation field.

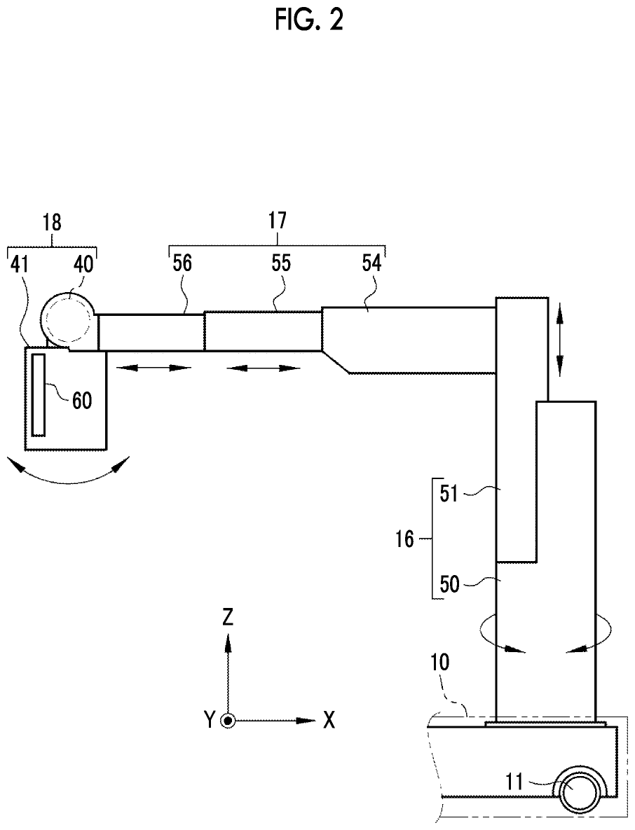

As shown in FIG. 2, the column unit 16 has a first column 50 and a second column 51. The first column 50 is provided on the upper surface of the carriage unit 10. The first column 50 can rotate around the Z axis with respect to the carriage unit 10. The second column 51 can move up and down along the Z-axis direction with respect to the first column 50.

The arm unit 17 has a fixed arm 54, a first arm 55, and the second arm 56. The fixed arm 54 is bent at a right angle with respect to the second column 51. The proximal end of the fixed arm 54 is attached to the second column 51. The first arm 55 is attached to the distal end of the fixed arm 54. That is, the fixed arm 54 connects the second column 51 and the first arm 55 to each other. The irradiation unit 18 is attached to the distal end of the second arm 56. The first arm 55 can move back and forth with respect to the fixed arm 54 along the bending direction of the fixed arm 54 perpendicular to the Z axis (in FIG. 2, an X-axis direction indicating the front-rear direction of the mobile radiographic imaging apparatus 2). The second arm 56 can move back and forth with respect to the first arm 55 along the bending direction of the fixed arm 54 perpendicular to the Z axis (in FIG. 2, the X-axis direction).

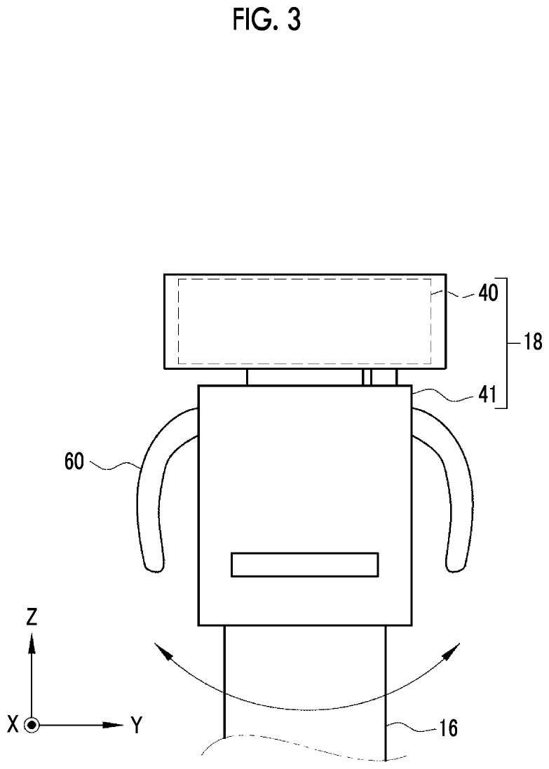

The irradiation unit 18 can rotate around an axis parallel to the width direction (in FIG. 2, the Y axis). As shown in FIG. 3, the irradiation unit 18 can rotate around an axis (in FIG. 2, the X axis) parallel to the front-rear direction.

A handgrip 60 is provided in the irradiation field limiter 41. The handgrip 60 is gripped by the operator OP in the case of moving the second arm 56 back and forth along the bending direction of the fixed arm 54 perpendicular to the Z axis. In addition, the handgrip 60 is gripped by the operator OP in the case of rotating the irradiation unit 18 around an axis parallel to the width direction and in the case of rotating the irradiation unit 18 around an axis parallel to the front-rear direction.

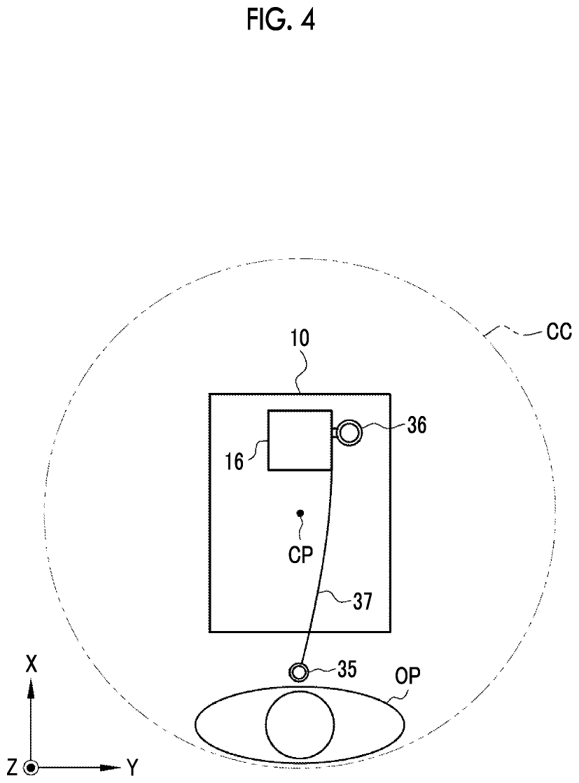

As simply shown in FIG. 4, the set range is defined by, for example, a circle CC drawn on the XY plane with a center position CP of the carriage unit 10 in the X-axis direction and the Y-axis direction as the center. The diameter of the circle CC is, for example, 1 in to 3m. The range in which the extension cable 37 can be extended is a range in which the operator OP present within the set range defined by the circle CC can come into contact with the second detection sensor 35.

As shown in FIG. 5, a first detection sensor 65 is provided in the whole front portion of the handle 22. The first detection sensor 65 detects the first operation of the operator OP. The first operation is an operation of the operator OP on the handle 22 and is an operation of the operator OP for switching from the automatic travel mode AM to the manual travel mode MM. The first detection sensor 65 detects that the operator OP has gripped the handle 22 as the first operation. More specifically, the first detection sensor 65 is a sensor that detects the contact of the hand of the operator OP with the handle 22. The first detection sensor 65 is, for example, a sensor that detects a hand contact based on the capacitance change or a sensor that detects a hand contact based on the temperature change. Alternatively, the first detection sensor 65 may be a mechanical lever switch that protrudes from the surface of the handle 22 and is turned off in a case where the handle 22 is not gripped by hand and is retracted into the handle 22 and is turned on in a case where the handle 22 is gripped by hand. In FIG. 5, the broken line indicates a state in which the handle 22 is gripped by both a right hand RH and a left hand LH of the operator OP.

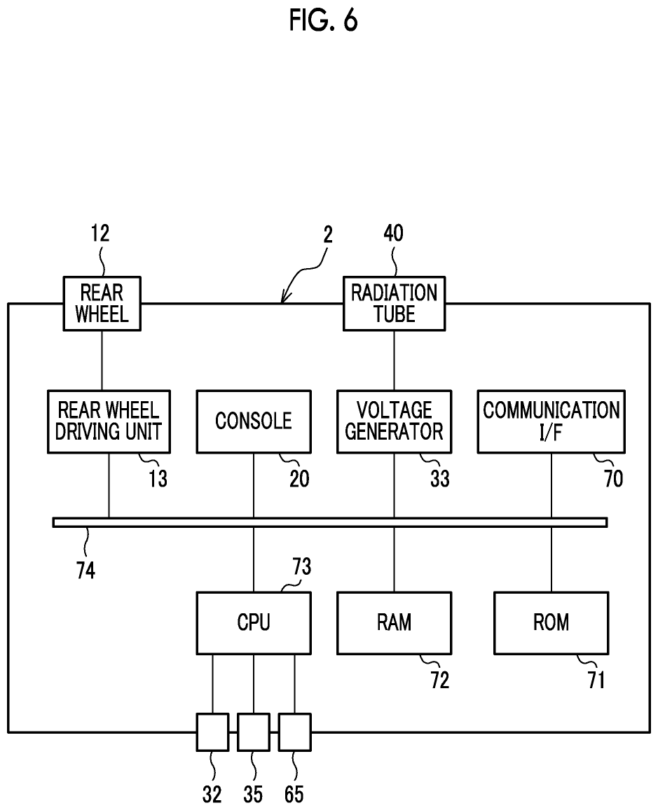

In FIG. 6, the mobile radiographic imaging apparatus 2 has a communication interface (I/F) 70, a read only memory (ROM) 71, a random access memory (RAM) 72, and a central processing unit (CPU) 73 in addition to the rear wheel driving unit 13, the console 20, and the voltage generator 33 described above. The rear wheel driving unit 13, the console 20, the voltage generator 33, the communication I/F 70, the ROM 71, the RAM 72, and the CPU 73 are connected to each other through a bus line 74. The ROM 71, the RAM 72, the CPU 73, and the bus line 74 are examples of a “computer” according to the technique of the present disclosure.

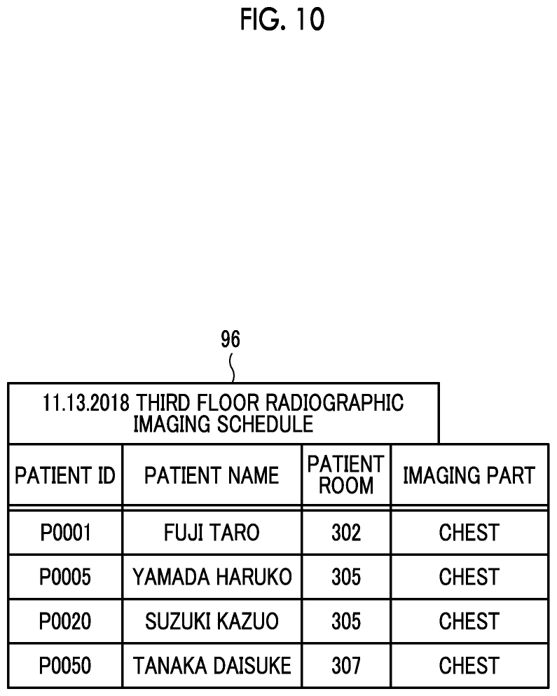

The communication I/F 70 includes a wireless communication interface for wireless communication with the electronic cassette 30. In addition, the communication I/F 70 includes a network interface for communicating with an external apparatus other than the electronic cassette 30 through a network. As an example of the external apparatus, there is a radiology information system (RIS) that manages information regarding radiographic imaging, such as imaging schedule information 96 (refer to FIGS. 8 and 10) indicating the schedule of radiographic imaging. Examples of the network include the Internet or a wide area network (WAN), such as a public communication network.

The ROM 71 stores various programs and various kinds of data added to the various programs. The RAM 72 is a work memory for the CPU 73 to execute processing. The CPU 73 reads a program stored in the ROM 71 to the RAM 72 and executes processing according to the read program. Therefore, the CPU 73 performs overall control of the operation of each unit of the mobile radiographic imaging apparatus 2.

The irradiation switch 32, the first detection sensor 65, and the second detection sensor 35 described above are connected to the CPU 73. The irradiation switch 32 outputs a warm-up command signal and an irradiation start command signal to the CPU 73. The first detection sensor 65 outputs a detection signal of the first operation to the CPU 73. The second detection sensor 35 outputs a detection signal of the second operation to the CPU 73. The detection signal of the first operation is an example of a “detection result of the first operation” according to the technique of the present disclosure. The detection signal of the second operation is an example of a “detection result of the second operation” according to the technique of the present disclosure.

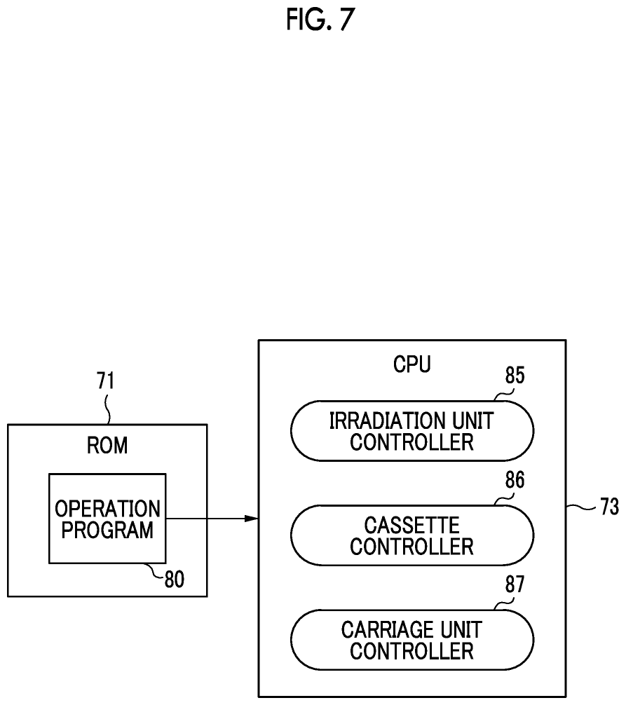

In FIG. 7, an operation program 80 is stored in the ROM 71. The operation program 80 is an example of an “operation program of a mobile radiographic imaging apparatus” according to the technique of the present disclosure. The CPU 73 executes the operation program 80 to function as an irradiation unit controller 85, a cassette controller 86, and a carriage unit controller 87 in cooperation with the RAM 72 and the like.

The irradiation unit controller 85 is a controller relevant to the irradiation unit 18. The irradiation unit controller 85 receives irradiation conditions input through the operation console 25, and sets the received irradiation conditions in the voltage generator 33. In addition, the irradiation unit controller 85 receives a warm-up command signal from the irradiation switch 32 and causes the radiation tube 40 to warn up. In addition, the irradiation unit controller 85 receives an irradiation start command signal from irradiation switch 32, and controls the operation of the voltage generator 33 to emit radiation from the radiation tube 40 under the set irradiation conditions.

The cassette controller 86 is a controller relevant to the electronic cassette 30. The cassette controller 86 controls the operation of the electronic cassette 30 by transmitting various control signals to the electronic cassette 30 through the communication I/F 70. The control signal transmitted to the electronic cassette 30 is, for example, a signal for giving an instruction for the accumulation of charges according to radiation in accordance with the irradiation start timing and a signal for reading the accumulated charges in accordance with the irradiation end timing. The cassette controller 86 receives a radiographic image from the electronic cassette 30 through the communication I/F 70. The cassette controller 86 performs control to display the acquired radiographic image on the display 26.

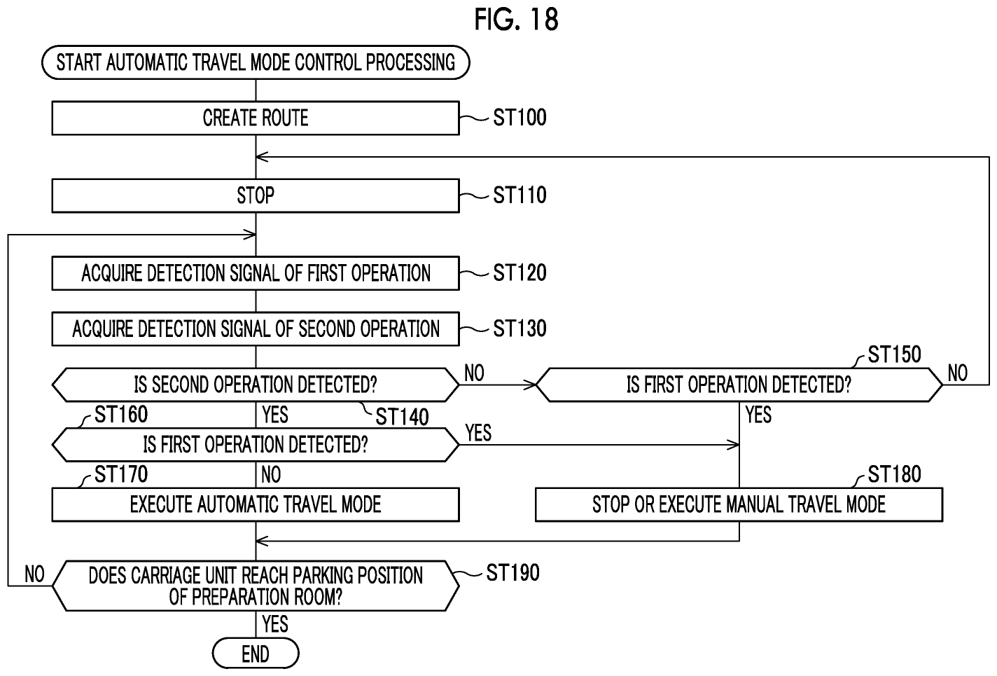

The carriage unit controller 87 is a controller relevant to the carriage unit 10. The carriage unit controller 87 executes automatic travel mode control processing (refer to FIG. 18) on the condition that the execution of the automatic travel mode AM has been selected through the operation console 25. In order to execute the automatic travel mode control processing, as shown in FIG. 8, the carriage unit controller 87 has a route creation unit 90, a first acquisition unit 91, a second acquisition unit 92, and a mode controller 93.

The route creation unit 90 creates a route, along which the carriage unit 10 travels, in the automatic travel mode AM. The route creation unit 90 reads the map information 95 and the imaging schedule information 96 from the ROM 71, and creates a route based on the map information 95 and the imaging schedule information 96 that have been read. The route creation unit 90 outputs the created route to the mode controller 93. The route along which the carriage unit 10 travels in the automatic travel mode AM may be manually set by the operator OP.

The first acquisition unit 91 acquires the detection signal of the first operation of the operator OP from the first detection sensor 65. The first acquisition unit 91 outputs the acquired detection signal of the first operation to the mode controller 93.

The second acquisition unit 92 acquires the detection signal of the second operation of the operator OP from the second detection sensor 35. The second acquisition unit 92 outputs the acquired detection signal of the second operation to the mode controller 93.

The mode controller 93 executes the automatic travel mode AM based on the route from the route creation unit 90. In addition, the mode controller 93 executes or stops the automatic travel mode AM or the manual travel mode MM according to the detection signal of the first operation from the first acquisition unit 91 and the detection signal of the second operation from the second acquisition unit 92.

As shown in FIG. 9, the map information 95 is information indicating the arrangement of patient rooms and the like on the floor in the hospital on which the mobile radiographic imaging apparatus 2 moves. FIG. 9 shows the map information 95 on the third floor as an example. There are a total of eight patient rooms of room 301, room 302, room 303, room 305, room 306, room 307, room 308, and room 310. In addition to these rooms, there are a preparation room, a treatment room, a nurse station, and the like.

The preparation room is a room where the mobile radiographic imaging apparatus 2 stands by. In the preparation room, the operator OP downloads the imaging schedule information 96 from the RIS and stores the imaging schedule information 96 in the ROM 71, or houses the electronic cassette 30 to be used in the cassette housing unit 21.

In the preparation room, a parking position PA of the carriage unit 10 is defined in advance. In each patient room, parking positions PB to PI of the carriage unit 10 are defined in advance. The parking position PA is a position outside the preparation room and corresponding to the entrance EA of the preparation room. The parking positions PB to PI are positions outside the patient rooms and corresponding to the entrances EB to EI of the patient rooms. Coordinates indicating the positions of the parking positions PA to PI on the floor are stored in the map information 95. From the coordinates, the distance between parking positions and the direction of each parking position can be known.

In the automatic travel mode AM, the parking position PA of the preparation room is the departure point and the last arrival point of the carriage unit 10. In the automatic travel mode AM, a position corresponding to the imaging schedule information 96 among the parking positions PB to PI is an intermediate point where the carriage unit 10 stops. The carrying-out of the carriage unit 10 from the preparation room to the parking position PA and the carrying-in of the carriage unit 10 from the parking position PA to the preparation room are performed by the manual travel mode MM. Similarly, the carrying-out of the carriage unit 10 from each patient room to the parking positions PB to PI and the carrying-in of the carriage unit 10 from the parking positions PB to PI to each patient room are performed by the manual travel mode MM.

The mode controller 93 stores a parking position reached by executing the automatic travel mode AM before carrying the carriage unit 10. After the carriage unit 10 is carried out, the mode controller 93 reads the next target parking position after the stored parking position from the route, and heads to the read next target parking position in the automatic travel mode AM.

In FIG. 10, information, such as patient identification data (ID), a patient name, a patient room, and an imaging part, is registered in the imaging schedule information 96. Here, the imaging schedule information 96 for the third floor on Nov. 13, 2018 is shown. Patient rooms for patients for whom radiographic imaging is scheduled are room 302, room 305, and room 307. In addition to these, information, such as patient's age, sex, disease name, and a bed position in the patient room, may be registered.

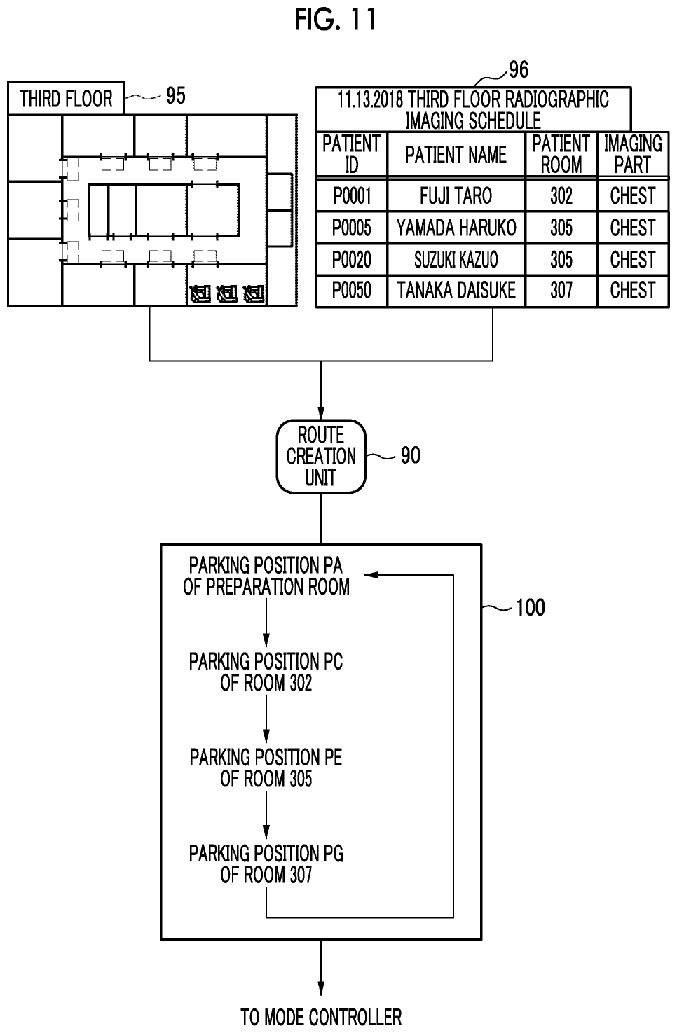

FIG. 11 shows a route 100 created by the route creation unit 90 in a case where the imaging schedule information 96 has the content shown in FIG. 10. In the imaging schedule information 96 shown in FIG. 10, as described above, room 302, room 305, and room 307 are patient rooms for patients for whom radiographic imaging is scheduled. For this reason, the route 100 is the content that the parking position PA of the preparation room is a departure point and a last arrival point and the carriage unit 10 moves in order from the parking position PC of the room 302 to the parking position PE of the room 305 and further to the parking position PG of the room 307. As described above, the parking position of the carriage unit 10 in the automatic travel mode is a position corresponding to the schedule of radiographic imaging, and includes a position outside a patient room that is defined in advance for each patient room.

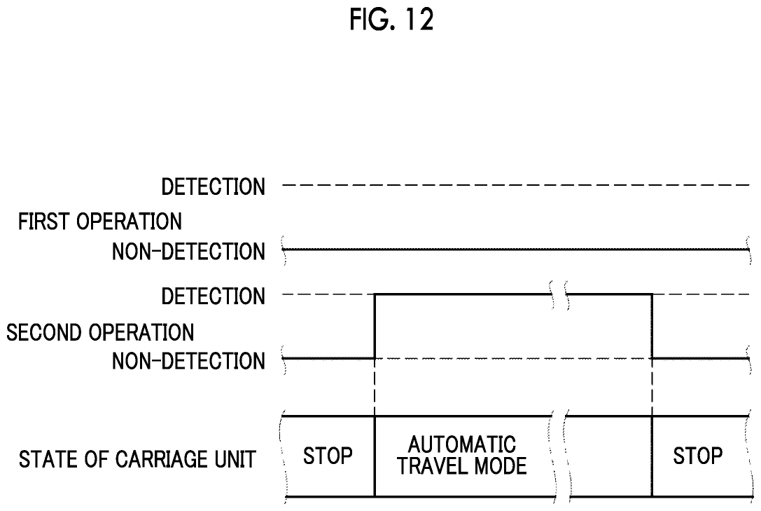

As shown in FIG. 12, in a case where neither the first operation nor the second operation is detected, the mode controller 93 stops the carriage unit 10. In a case where the second operation is detected in a state in which neither the first operation nor the second operation is detected, the mode controller 93 executes the automatic travel mode AM. In a case where the first operation is not detected and the second operation is detected, the mode controller 93 continues the execution of the automatic travel mode AM while the second operation is detected.

In a case where the second operation is not detected in a state in which the automatic travel mode AM is executed, the mode controller 93 stops the automatic travel mode AM, that is, stops the carriage unit 10. In the present embodiment, the case where the second operation is not detected in a state in which the automatic travel mode AM is executed means that one hand of the operator OP has separated from the second detection sensor 35. The case where one hand of the operator OP has separated from the second detection sensor 35 includes a case where the operator OP has separated up to a position outside the set range.

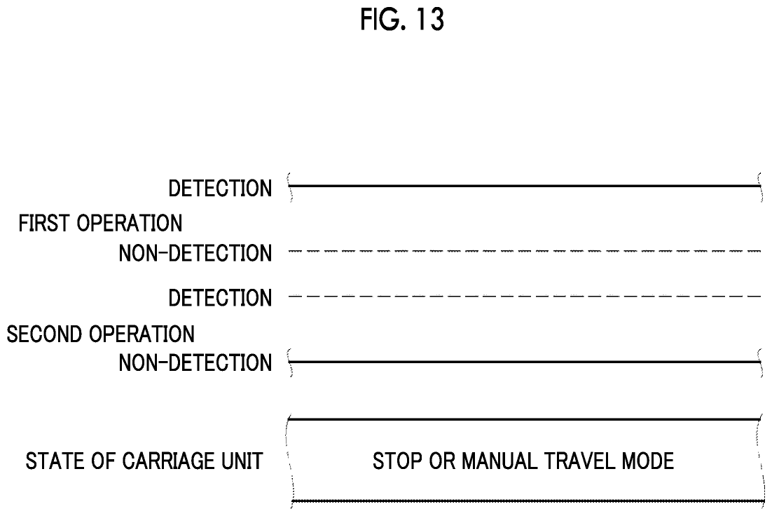

As shown in FIG. 13, in a case where the first operation is detected and the second operation is not detected, the mode controller 93 stops the carriage unit 10 or executes the manual travel mode MM.

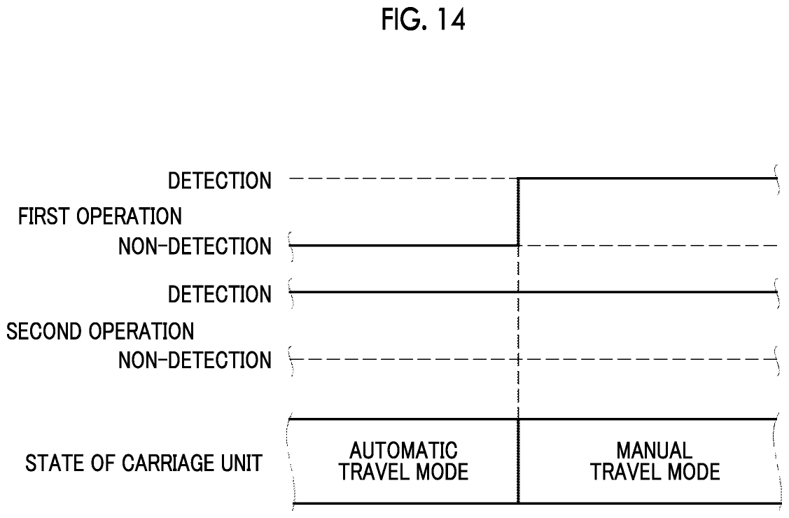

As shown in FIG. 14, in a case where the first operation is detected while the second operation is detected and the automatic travel mode AM is executed, the mode controller 93 stops the automatic travel mode AM and executes the manual travel mode MM.

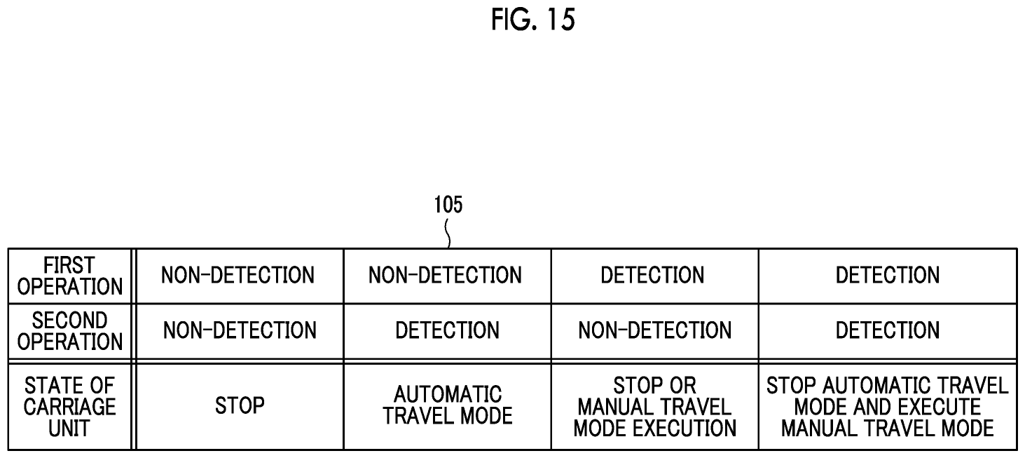

The above-described content of FIGS. 12 to 14 are summarized as shown in Table 105 of FIG. 15. That is, in a case where neither the first operation nor the second operation is detected, the mode controller 93 stops the carriage unit 10. In a case where the first operation is not detected and the second operation is detected, the mode controller 93 executes the automatic travel mode AM. In a case where the first operation is detected and the second operation is not detected, the mode controller 93 stops the carriage unit 10 or executes the manual travel mode MM. In a case where both the first operation and the second operation are detected, the mode controller 93 stops the automatic travel mode AM and executes the manual travel mode MM.

The state in which the automatic travel mode AM is executed is, specifically, a state shown in FIG. 16. That is, the state in which the automatic travel mode AM is executed is a state in which the right hand RH of the operator OP is in contact with the second detection sensor 35 extended by the extension cable 37. In addition, the state in which the automatic travel mode AM is executed is a state in which the operator OP accompanies the automatic traveling of the mobile radiographic imaging apparatus 2 without gripping the handle 22 by the left hand LH.

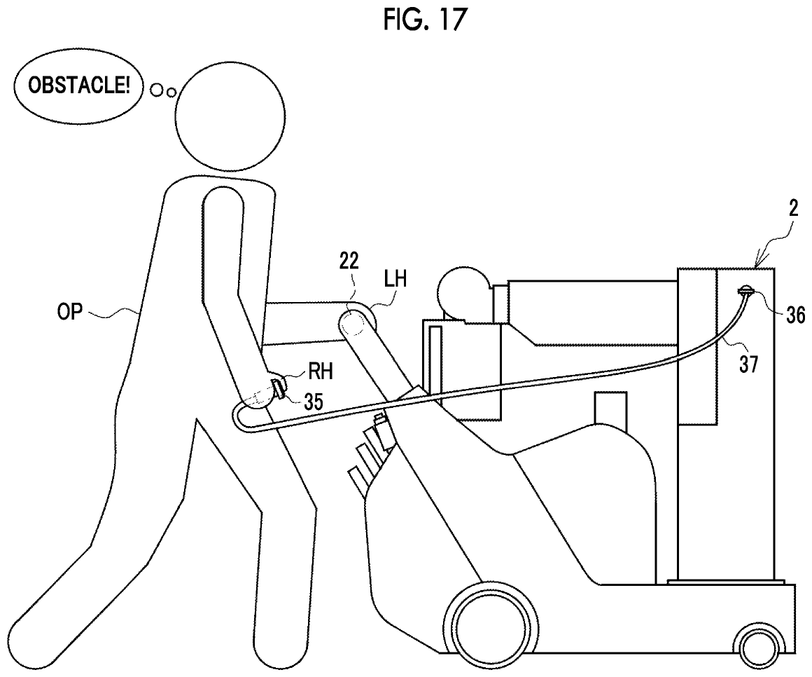

The state in which the automatic travel mode AM is stopped and the manual travel mode MM is executed is, specifically, a state shown in FIG. 17. That is, the state in which the automatic travel mode AM is stopped and the manual travel mode MM is executed is a state in which the operator OP grips the handle 22 with the left hand LH while bringing the right hand RH into contact with the second detection sensor 35. As a situation in which it is necessary to switch from the automatic travel mode AM to the manual travel mode MM as described above, there is a case where an obstacle, such as a person or a food distribution car, suddenly crosses before the mobile radiographic imaging apparatus 2.

Next, the operation based on the above configuration will be described with reference to the flowchart shown in FIG. 18. In FIG. 18, the automatic travel mode control processing executed by the CPU 73 in accordance with the operation program 80 on the condition that the execution of the automatic travel mode AM has been selected through the operation console 25 will be described. The automatic travel mode control processing is processing for causing the CPU 73 to function as the carriage unit controller 87 shown in FIG. 8, that is, the route creation unit 90, the first acquisition unit 91, the second acquisition unit 92, and the mode controller 93.

First, as shown in FIG. 11, the route creation unit 90 creates a route, along which the carriage unit 10 travels in the automatic travel mode AM, based on the map information 95 and the imaging schedule information 96 stored in the ROM 71 (step ST100). The created route is output from the route creation unit 90 to the mode controller 93.

The carriage unit 10 is stopped by the mode controller 93 (step ST110). The first acquisition unit 91 acquires the detection signal of the first operation from the first detection sensor 65 (step ST120). The second acquisition unit 92 acquires the detection signal of the second operation from the second detection sensor 35 (step ST130). Step ST120 is an example of “first acquisition step” according to the technique of the present disclosure. Step ST130 is an example of “second acquisition step” according to the technique of the present disclosure.

In a case where neither the first operation nor the second operation is detected (NO in both step ST140 and step ST150), as shown in FIG. 12, the carriage unit 10 remains stopped by the mode controller 93 (step ST110). In a case where the second operation is detected (YES in step ST140) and the first operation is not detected (NO in step ST160), as shown in FIGS. 12, 16, and the like, the mode controller 93 executes the automatic travel mode AM based on the route created by the route creation unit 90, so that the carriage unit 10 travels in the automatic travel mode AM (step ST170).

In a case where the second operation is not detected (NO in step ST140) and the first operation is detected (YES in step ST150), as shown in FIG. 13, the carriage unit 10 is stopped by the mode controller 93. Alternatively, the manual travel mode MM is executed by the mode controller 93, and the carriage unit 10 travels in the manual travel mode MM (step ST180).

In a case where both the first operation and the second operation are detected (YES in both step ST140 and step ST160), as shown in FIGS. 14 and 17, the automatic travel mode AM is stopped and the manual travel mode MM is executed by the mode controller 93, and the carriage unit 10 travels in the manual travel mode MM (step ST180). Steps ST170 and ST180 are an example of “mode control step” according to the technique of the present disclosure.

The processing from step ST120 to step ST170 or step ST180 is repeated until the carriage unit 10 reaches the parking position PA of the preparation room that is a last arrival point (YES in step ST190).

As described above, the mobile radiographic imaging apparatus 2 comprises the carriage unit 10, the handle 22, the first detection sensor 65, the second detection sensor 35, and the mode controller 93. The first detection sensor 65 detects the first operation of the operator OP, and the second detection sensor 35 detects the second operation of the operator OP. The first operation is an operation of the operator OP on the handle 22, and is an operation for switching from the automatic travel mode AM in which the carriage unit 10 travels without an operation of the operator OP on the handle 22 to the manual travel mode MM in which the carriage unit 10 travels by an operation of the operator OP on the handle 22. The second operation is an operation different from the first operation, and is an operation performed within a set range that is set in advance around the carriage unit 10. The second operation is a continuous operation for continuing the execution of the automatic travel mode AM. In a case where the first operation is not detected by the first detection sensor 65 and the second operation is detected by the second detection sensor 35, the mode controller 93 continues the execution of the automatic travel mode AM. In a case where the first operation is detected by the first detection sensor 65 during the execution of the automatic travel mode AM, the mode controller 93 stops the automatic travel mode AM and executes the manual travel mode MM.

Since the second operation is an operation performed within the set range that is set in advance around the carriage unit 10, the operator OP is necessarily present within the set range during the execution of the automatic travel mode AM. In a case where the first operation is detected by the first detection sensor 65 during the execution of the automatic travel mode AM, the automatic travel mode AM is stopped and the manual travel mode MM is executed. Accordingly, the automatic travel mode AM can be immediately stopped as long as the operator OP performs the first operation. Therefore, it is possible to realize a safer automatic travel mode.

In a case where the second operation is not detected by the second detection sensor 35, the mode controller 93 stops the automatic travel mode AM. In other words, the mode controller 93 executes the automatic travel mode AM only in a case where the second operation is detected. For this reason, the execution of the automatic travel mode AM is allowed only in an environment in which the eyes of the operator OP reach and the operator OP can perform the second operation. Therefore, it is possible to realize a safer automatic travel mode.

The first detection sensor 65 detects that the operator OP has gripped the handle 22 as the first operation. More specifically, the first detection sensor 65 is a sensor that detects the contact of the hand of the operator OP with the handle 22. The act of the operator OP for bringing his or her hand into contact with the handle 22 to grip the handle 22 is an expression of the intention that the operator OP himself or herself desires to steer the carriage unit 10. Therefore, switching to the manual travel mode MM reflecting the intention of the operator OP is possible.

The second detection sensor 35 is provided in the upper portion of the column unit 16 that is a portion other than the handle 22, and detects that one hand of the operator OP has come into contact with the second detection sensor 35 itself as the second operation. Therefore, since there is no possibility that one of the first operation and the second operation is erroneously detected as the other operation, the carriage unit 10 can be made to travel while accurately reflecting the intention of the operator OP. In addition, in the case of providing the second detection sensor 35 in the known mobile radiographic imaging apparatus 2, the modification can be made with relatively little time and effort.

The second detection sensor 35 is attached to one end of the extension cable 37, and can be used by being detached from the upper portion of the column unit 16. Compared with a case where the second detection sensor 35 is fixed to the upper portion of the column unit 16, the degree of freedom of the position of the second detection sensor 35 is high. Therefore, the operator OP can easily perform the second operation.

The parking position of the carriage unit 10 in the automatic travel mode AM is a position corresponding to the schedule of radiographic imaging, and includes a position outside a patient room that is defined in advance for each patient room. Therefore, the safety of the patient in the patient room can be ensured without the mobile radiographic imaging apparatus 2 executing the automatic travel mode AM being moved into the patient room.

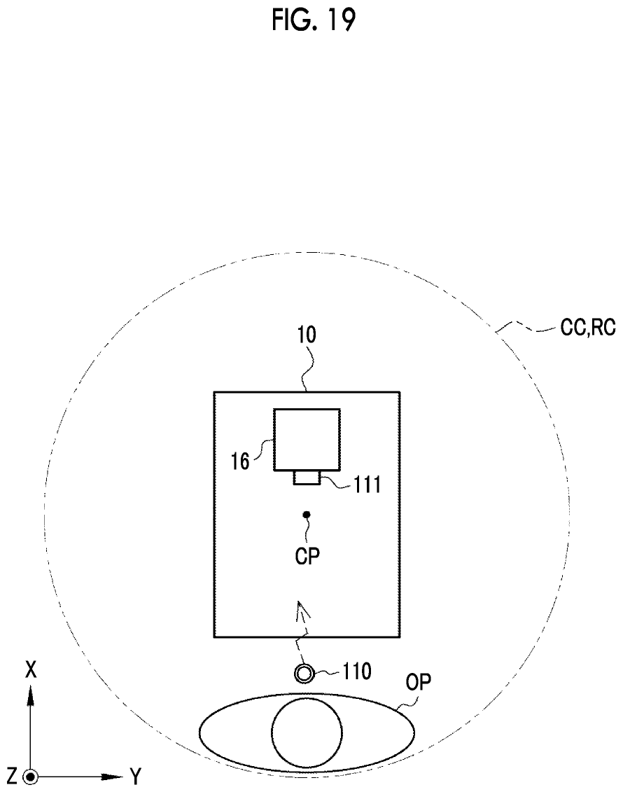

As shown in FIG. 19, a second detection sensor 110 having a wireless transmission function may be used. In this case, for example, a wireless receiver 111 is provided on the surface of the column unit 16 on the rear side of the carriage unit 10. The second detection sensor 110 and the wireless receiver 111 wirelessly transmit and receive the detection result of the second operation within a communication range RC. The communication range RC is the same range as the set range defined by the circle CC.

Thus, the degree of freedom of the position of the second detection sensor 35 can be further increased by using the second detection sensor 110 having a wireless transmission function for wirelessly transmitting the detection result of the second operation within the communication range RC limited by the set range. The operator OP can perform the second operation more easily.

The second detection sensor may be a wireless transmitter that periodically emits a wireless signal. In this case, the second operation is an operation in which the operator OP having a second detection sensor, which is a wireless transmitter, moves into the communication range RC.

The second detection sensor may be provided on the operation console 25 or may be provided integrally with the irradiation switch 32. Alternatively, the second detection sensor may be provided on the side surface of the central unit 15 next to the handle 22.

The center of the circle CC specifying the set range may be the center position of the handle 22 instead of the center position CP of the carriage unit 10.

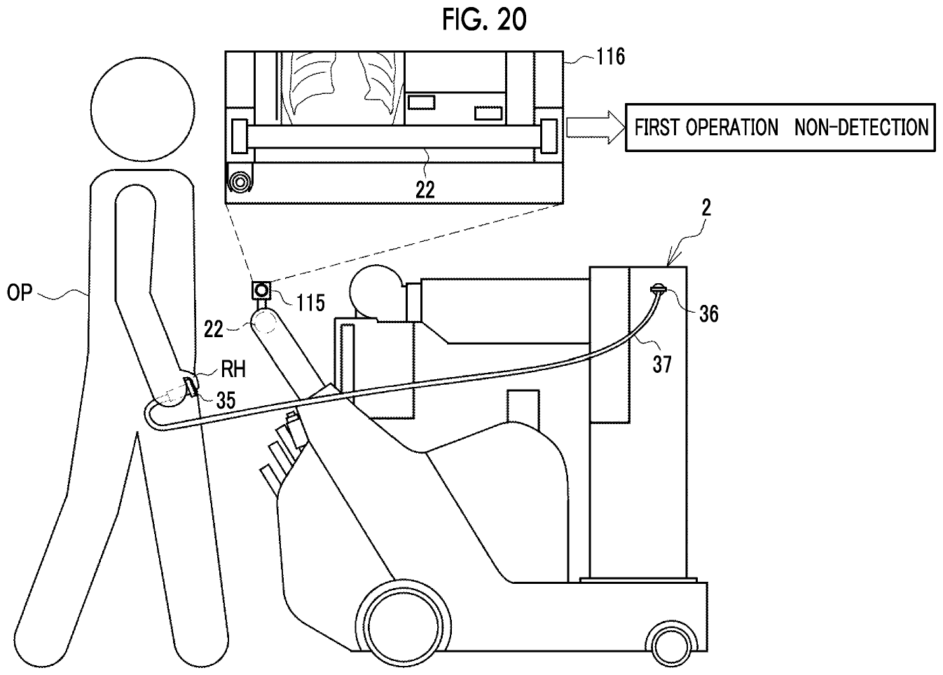

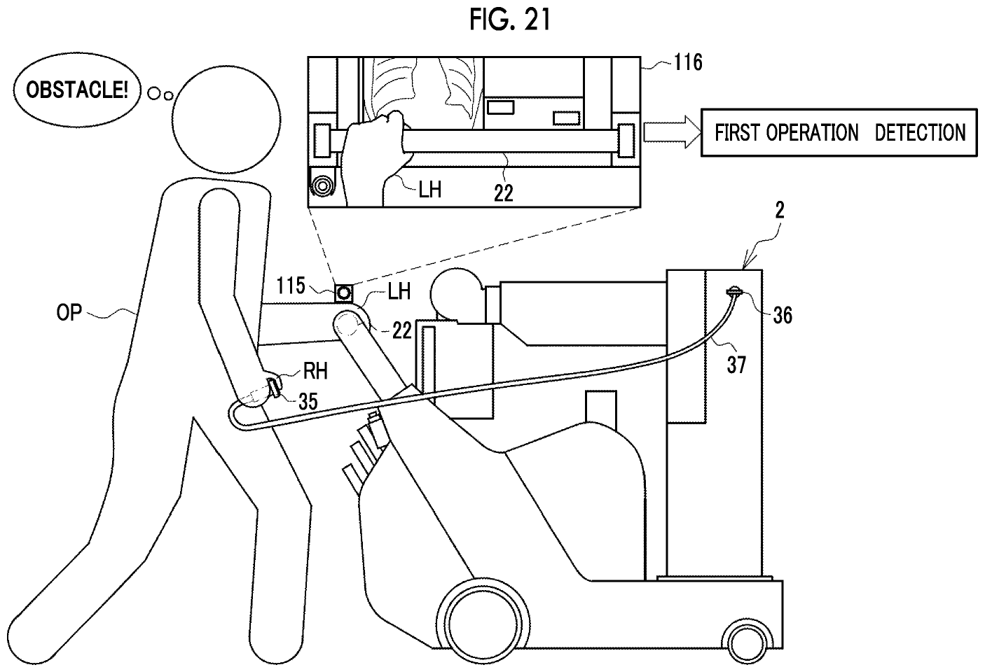

As shown in FIGS. 20 and 21, a first detection sensor 115 configured by a camera may be used. The first detection sensor 115 is attached to the left upper portion of the handle 22. The first detection sensor 115 images a state in which the handle 22 is gripped by the operator OP. In this case, the mode controller 93 performs image recognition of an image 116 captured by the first detection sensor 115. Then, in a case where the hand of the operator OP gripping the handle 22 is not reflected in the image 116, the mode controller 93 determines that the first operation has not been detected (non-detection of the first operation). On the other hand, in a case where the hand of the operator OP gripping the handle 22 is reflected in the image 116, the mode controller 93 determines that the first operation has been detected.

FIG. 20 shows a state in which the automatic travel mode AM is being executed as in the state shown in FIG. 16. The operator OP is not gripping the handle 22 with the left hand LH. For this reason, the left hand LH is not reflected in the image 116. In this case, therefore, the mode controller 93 determines that the first operation has not been detected, and the automatic travel mode AM is executed.

FIG. 21 shows a state in which the automatic travel mode AM is stopped and the manual travel mode MM is executed as in the state shown in FIG. 17. The operator OP grips the handle 22 with the left hand LH. For this reason, the left hand LH gripping the handle 22 is reflected in the image 116. In this case, therefore, the mode controller 93 determines that the first operation has been detected, and the automatic travel mode AM is switched to the manual travel mode MM.

As described above, since the first detection sensor 115 configured by a camera for imaging the gripping state of the handle 22 by the operator OP is used, the first operation can be detected based on the actual gripping state of the handle 22. Therefore, it is possible to distinguish between the first operation and the contact of the hand with the handle 22 that is not intended for the steering of the carriage unit 10, such as a momentary contact of the hand with the handle 22.

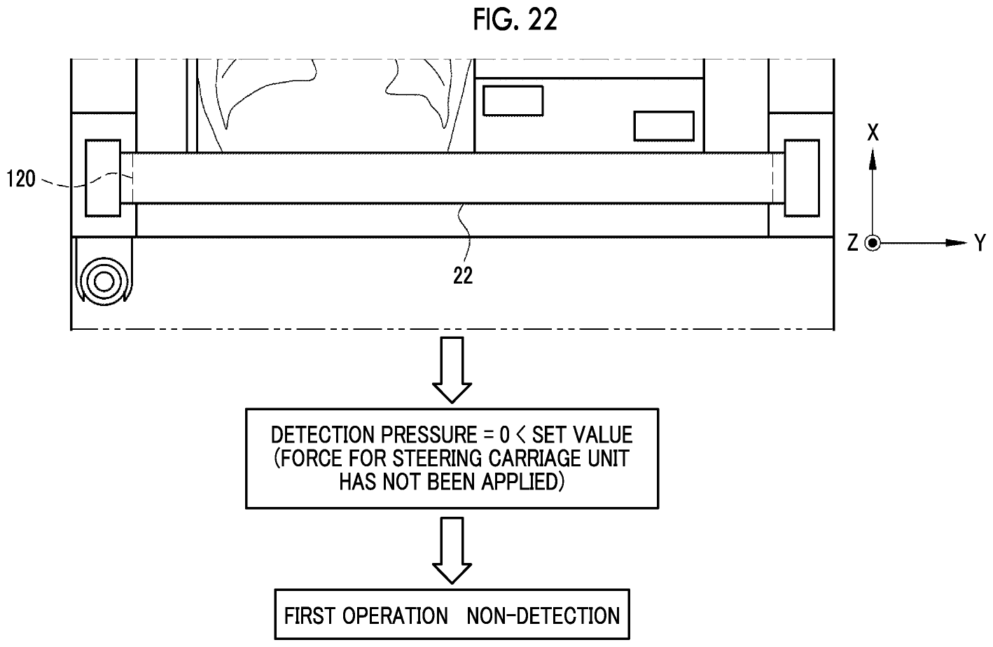

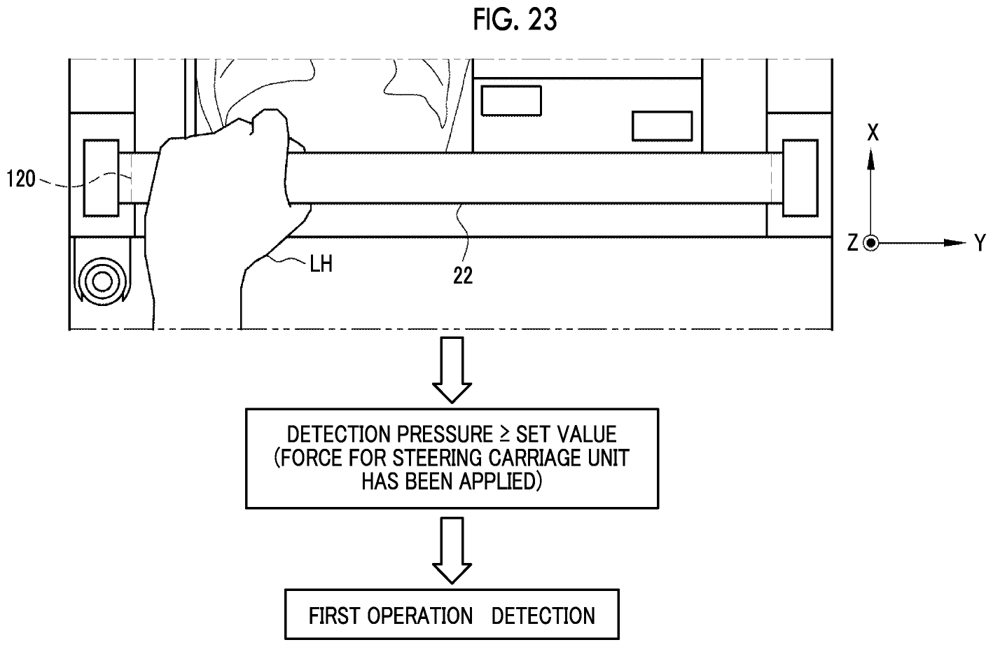

As shown in FIGS. 22 and 23, a first detection sensor 120 configured by a pressure sensor may be used. The first detection sensor 120 is provided on the entire surface of the handle 22. The first detection sensor 120 detects that a force for steering the carriage unit 10 has been applied to the handle 22 by detecting the pressure applied to the handle 22 by the hand of the operator OP. In this case, the mode controller 93 performs magnitude comparison between the pressure detected by the first detection sensor 120 and a set value set in advance. Then, in a case where the pressure detected by the first detection sensor 120 is lower than the set value, the mode controller 93 determines that the force for steering the carriage unit 10 has not been applied and accordingly the first operation has not been detected (non-detection of the first operation). On the contrary, in a case where the pressure detected by the first detection sensor 120 is equal to or greater than the set value, the mode controller 93 determines that the force for steering the carriage unit 10 has been applied and accordingly the first operation has been detected.

FIG. 22 shows a state in which the automatic travel mode AM is being executed as in the state shown in FIGS. 16 and 20. The operator OP is not gripping the handle 22 with the left hand LH. For this reason, the pressure detected by the first detection sensor 120 is 0, which is lower than the set value. In this case, therefore, the mode controller 93 determines that the first operation has not been detected, and the automatic travel mode AM is executed.

FIG. 23 shows a state in which the automatic travel mode AM is stopped and the manual travel mode MM is executed as in the state shown in FIGS. 17 and 21. The operator OP grips the handle 22 with the left hand LH. Then, the pressure detected by the first detection sensor 120 is equal to or greater than the set value. In this case, therefore, the mode controller 93 determines that the first operation has been detected, and the automatic travel mode AM is switched to the manual travel mode MM.

As described above, since the first detection sensor 120 configured by a pressure sensor for detecting that a force for steering the carriage unit 10 has been applied to the handle 22 is used, the first operation can be detected based on the actual gripping state of the handle 22 as in the case of the first detection sensor 115. Then, it is possible to distinguish between the first operation and the contact of the hand with the handle 22 that is not intended for the steering of the carriage unit 10.

A piezoelectric sensor that detects the force applied to the carriage unit 10 by the operator OP may be used as the first detection sensor for detecting that a force for steering the carriage unit 10 has been applied to the handle 22.

The first detection sensor may be at least one of a sensor for detecting the contact of the hand of the operator OP with the handle 22 (first detection sensor 65), a camera for imaging the gripping state of the handle 22 by the operator OP (first detection sensor 115), or a sensor for detecting that a force for steering the carriage unit 10 has been applied to the handle 22 (first detection sensor 120). That is, these sensors may be used in combination. For example, the first detection sensor 65 and the first detection sensor 115 are used in combination. Then, only in a case where the contact of the hand of the operator OP is detected by the first detection sensor 65 and the hand of the operator OP gripping the handle 22 is reflected in the image 116 captured by the first detection sensor 115, the mode controller 93 determines that the first operation has been detected. In this manner, it is possible to further reduce the probability of erroneously detecting the first operation.

### Second Embodiment

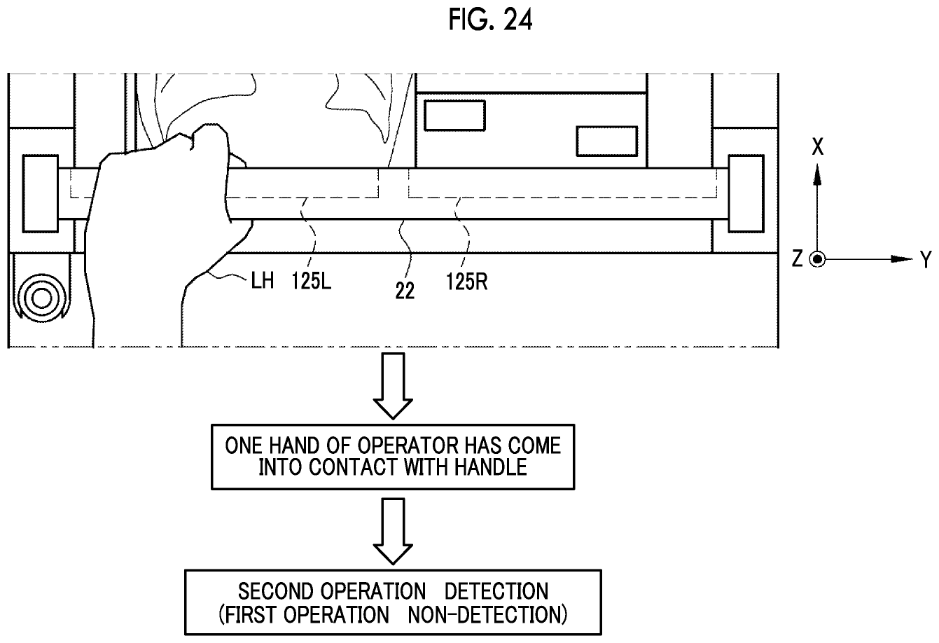

In a second embodiment shown in FIGS. 24 to 30, it is detected as the first operation that the operator OP has gripped the handle 22 with both hands, and it is detected as the second operation that one hand of the operator OP has come into contact with the handle 22.

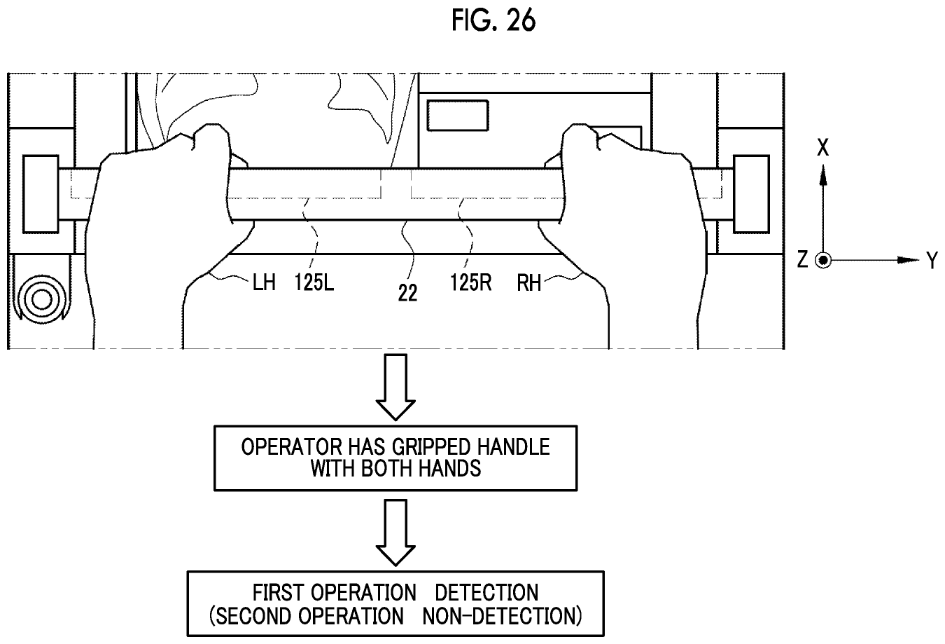

As shown in FIGS. 24 and 26, a left hand sensor 125L and a right hand sensor 125R are provided in the handle 22. The left hand sensor 125L is provided in the whole front left portion of the handle 22. The right hand sensor 125R is provided in the whole front right portion of the handle 22. The left hand sensor 125L is a sensor that detects the contact of the left hand LH of the operator OP with the handle 22. The right hand sensor 125R is a sensor that detects the contact of the right hand RH of the operator OP with the handle 22. The left hand sensor 125L and the right hand sensor 125R are, for example, a sensor that detects a hand contact based on the capacitance change, a sensor that detects a hand contact based on the temperature change, or a mechanical lever switch.

In a case where one of the left hand sensor 125L and the right hand sensor 125R detects that the hand of the operator OP has come into contact with the handle 22, the mode controller 93 determines that the second operation has been detected. In other words, the mode controller 93 determines that the first operation has not been detected (non-detection of the first operation). In a case where both the left hand sensor 125L and the right hand sensor 125R detect that the hand of the operator OP has come into contact with the handle 22, the mode controller 93 determines that the operator OP has gripped the handle 22 with both hands and accordingly the first operation has been detected. In other words, the mode controller 93 determines that the second operation has not been detected (non-detection of the second operation). That is, one the left hand sensor 125L or the right hand sensor 125R is an example of a “second detection sensor” according to the technique of the present disclosure. In addition, both the left hand sensor 125L and the right hand sensor 125R are examples of a “first detection sensor” according to the technique of the present disclosure.

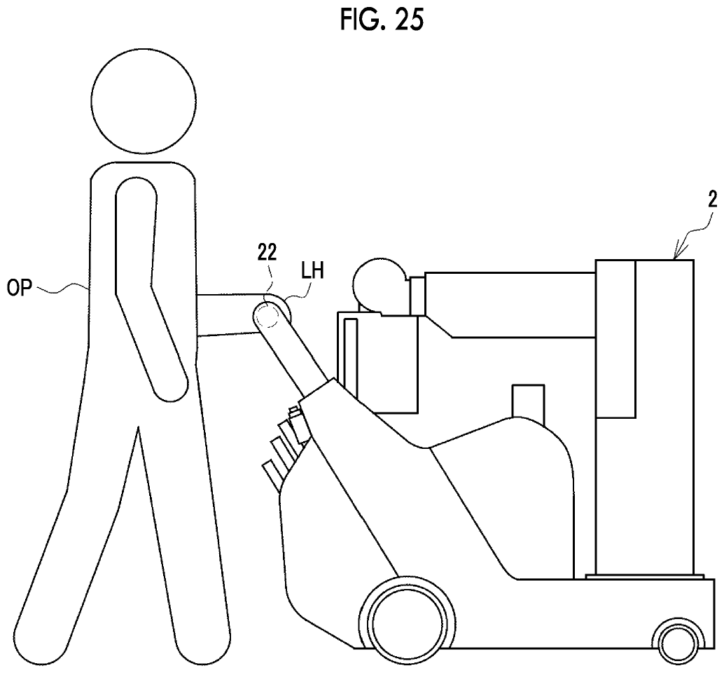

FIGS. 24 and 25 show a state in which the automatic travel mode AM is being executed. The operator OP grips the handle 22 with the left hand LH, but does not grip the handle 22 with the right hand RH. For this reason, only the left hand sensor 125L detects that the hand of the operator OP has come into contact with the handle 22. In this case, therefore, the mode controller 93 determines that the second operation has been detected, and the automatic travel mode AM is executed.

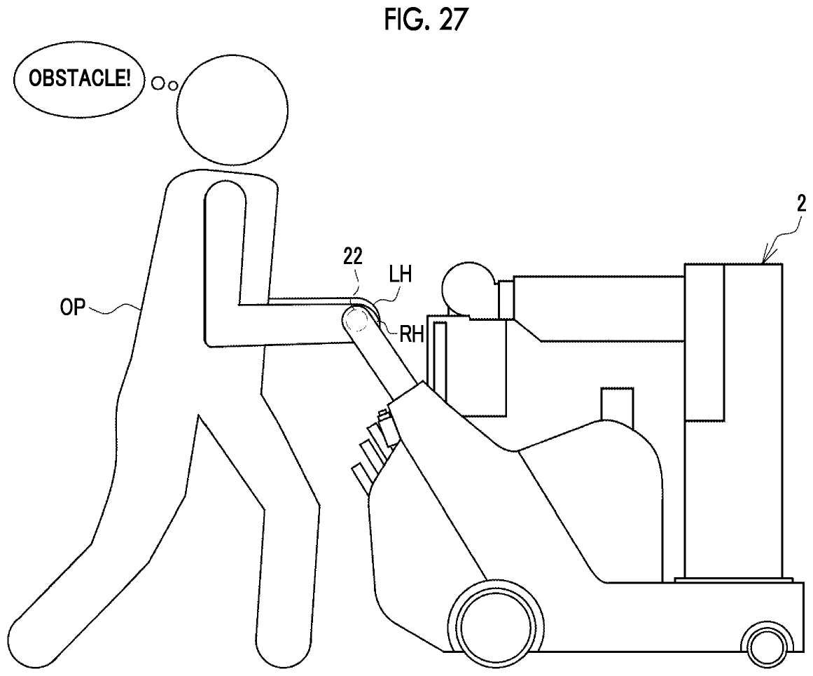

FIGS. 26 and 27 show a state in which the automatic travel mode AM is stopped and the manual travel mode MM is executed. The operator OP grips the handle 22 with both the left hand LH and the right hand RH. For this reason, both the left hand sensor 125L and the right hand sensor 125R detect that the hand of the operator OP has come into contact with the handle 22. In this case, therefore, the mode controller 93 determines that the first operation has been detected, and the automatic travel mode AM is switched to the manual travel mode MM.

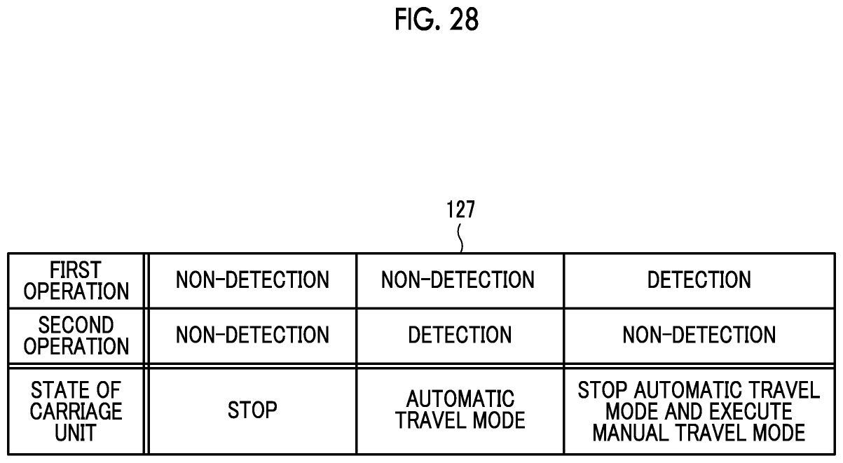

In the second embodiment, the state of the carriage unit 10 for detection and non-detection of the first operation and detection and non-detection of the second operation is shown in Table 127 of FIG. 28. That is, in a case where neither the first operation nor the second operation is detected, the mode controller 93 stops the carriage unit 10. In a case where the first operation is not detected and the second operation is detected, the mode controller 93 executes the automatic travel mode AM. In a case where the first operation is detected and the second operation is not detected, the mode controller 93 stops the automatic travel mode AM and executes the manual travel mode MM. In the second embodiment, there is no case where both the first operation and the second operation are detected.

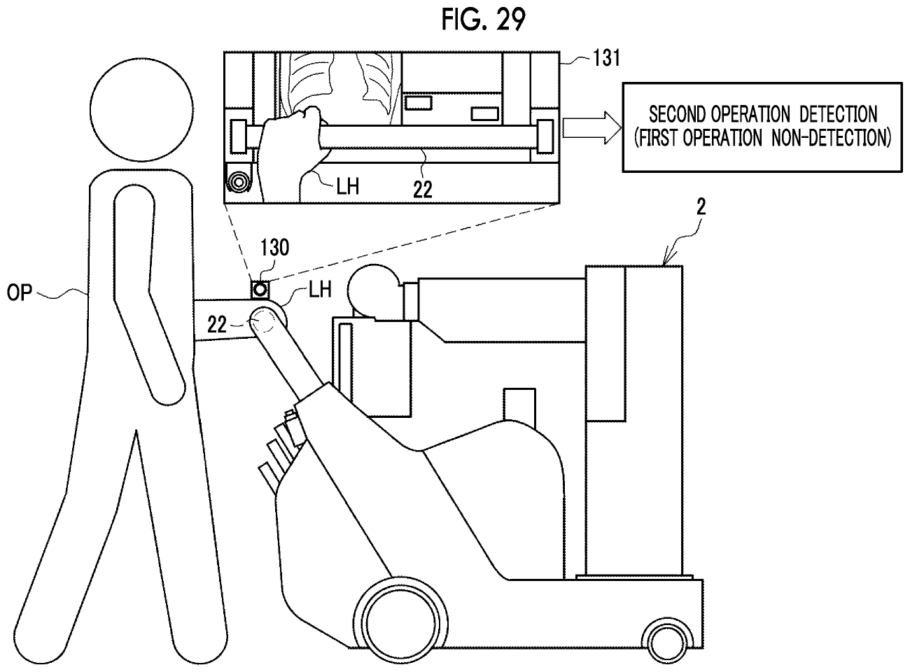

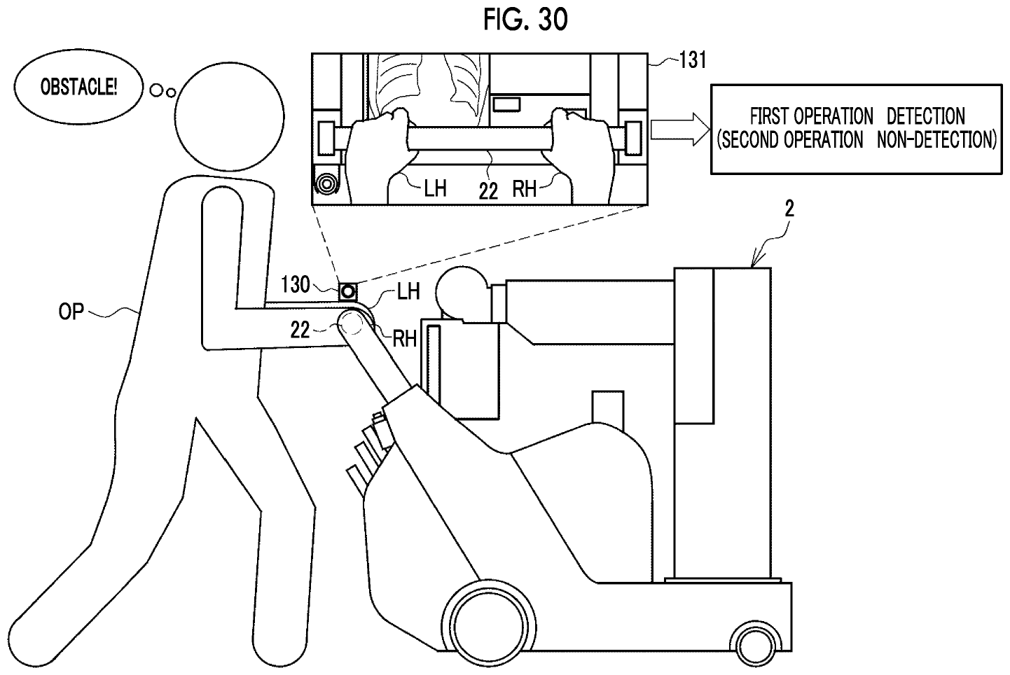

As shown in FIGS. 29 and 30, a first and second detection sensor 130 configured by a camera for imaging the gripping state of the handle 22 by the operator OP, such as the first detection sensor 115 shown in FIGS. 20 and 21 in the first embodiment described above, may be used. The first and second detection sensor 130 is an example of “a first detection sensor and a second detection sensor” according to the technique of the present disclosure. In a case where only one hand of the operator OP gripping the handle 22 is reflected in an image 131 captured by the first and second detection sensor 130, the mode controller 93 determines that the second operation has been detected. In other words, the mode controller 93 determines that the first operation has not been detected (non-detection of the first operation). On the other hand, in a case where both the hands of the operator OP gripping the handle 22 are reflected in the image 131 captured by the first and second detection sensor 130, the mode controller 93 determines that the first operation has been detected. In other words, the mode controller 93 determines that the second operation has not been detected (non-detection of the second operation).

FIG. 29 shows a state in which the automatic travel mode AM is being executed as in the state shown in FIGS. 24 and 25. The operator OP grips the handle 22 with the left hand LH, but does not grip the handle 22 with the right hand RH. For this reason, only the left hand LH is reflected in the image 131. In this case, therefore, the mode controller 93 determines that the second operation has been detected, and the automatic travel mode AM is executed.

FIG. 30 shows a state in which the automatic travel mode AM is stopped and the manual travel mode MM is executed as in the state shown in FIGS. 26 and 27. The operator OP grips the handle 22 with the left hand LH and the right hand RH. For this reason, the left hand LH and the right hand RH gripping the handle 22 are reflected in the image 131. In this case, therefore, the mode controller 93 determines that the first operation has been detected, and the automatic travel mode AM is switched to the manual travel mode MM.

In this manner, by using the first and second detection sensor 130 configured by a camera for imaging the gripping state of the handle 22 by the operator OP, the same effect as in the case of the first detection sensor 115 can be obtained.

As in the case of the first embodiment described above, the left hand sensor 125L and the right hand sensor 125R may be used in combination with the first and second detection sensor 130. In this case, only in a case where the contact of both the hands of the operator OP is detected by the left hand sensor 125L and the right hand sensor 125R and both the hands of the operator OP gripping the handle 22 are reflected in the image 131 captured by the first and second detection sensor 130, the mode controller 93 determines that the first operation has been detected.

As described above, in the second embodiment, it is detected as the first operation that the operator OP has gripped the handle 22 with both hands, and it is detected as the second operation that one hand of the operator OP has come into contact with the handle 22. Therefore, the first detection sensor and the second detection sensor can be disposed so as to be concentrated on the handle 22, and this can contribute to downsizing of the mobile radiographic imaging apparatus 2.

### Third Embodiment

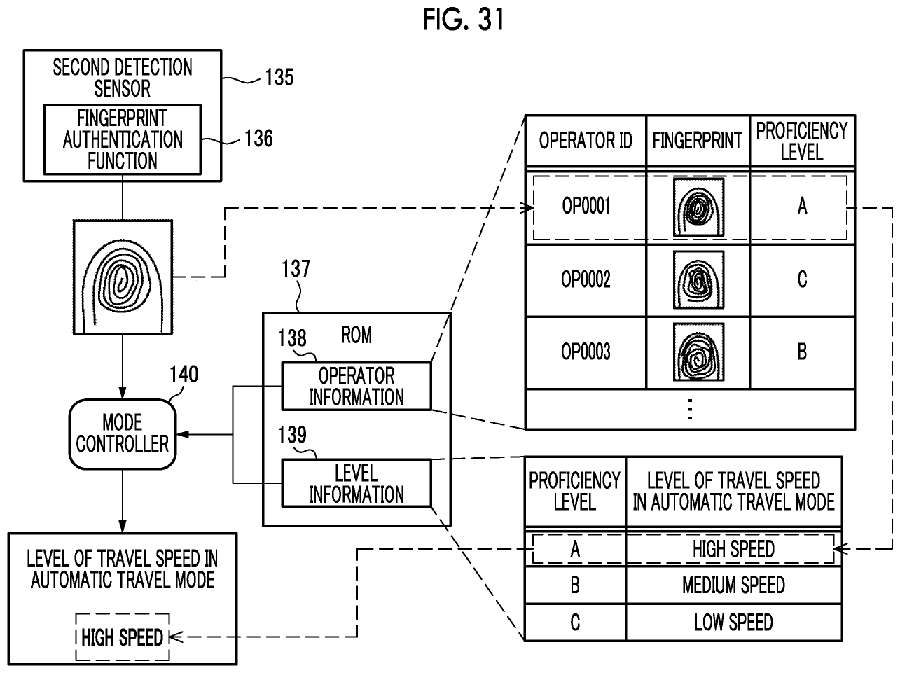

In a third embodiment shown in FIG. 31, a second detection sensor having an identification function for identifying the operator OP is used, and the level of the automatic travel mode AM is changed according to the identification result of the operator OP by the identification function.

In FIG. 31, a second detection sensor 135 has a fingerprint authentication function 136 for authenticating the fingerprint of the finger of the hand of the operator OP that has come into contact with the second detection sensor 135 itself. The fingerprint authentication function 136 is an example of an “identification function” according to the technique of the present disclosure.

Operator information 138 and level information 139 are stored in the ROM 137. In the operator information 138, a fingerprint and a proficiency level are registered for each operator ID for identifying each operator OP. The proficiency level is literally an index indicating the proficiency level of the operation of the mobile radiographic imaging apparatus 2. The proficiency level is three levels of A, B, and C, with A being the highest, B being the middle, and C being the lowest. The proficiency level is set by a supervisor of the operator OP, for example.

In the level information 139, the level of the travel speed in the automatic travel mode AM is registered for each proficiency level. That is, a high speed is registered in the case of the proficiency level A, a medium speed is registered in the case of the proficiency level B, and a low speed is registered in the case of the proficiency level C. The level of the travel speed in the automatic travel mode AM is an example of a “level of automatic travel mode” according to the technique of the present disclosure.

A mode controller 140 receives the fingerprint of the operator OP authenticated by the fingerprint authentication function 136 from the second detection sensor 135. The mode controller 140 compares the received fingerprint with a fingerprint registered in the operator information 138 to identify the operator OP. The mode controller 140 derives the level of the travel speed in the automatic travel mode AM according to the proficiency level of the identified operator OP from the level information 139. The mode controller 140 changes the level of the travel speed in the automatic travel mode AM to the derived level. FIG. 31 shows an example in which the proficiency level of the identified operator OP is A and the level of the travel speed in the automatic travel mode AM is changed to high speed.

As described above, in the third embodiment, the second detection sensor 135 having an identification function for identifying the operator OP is used, and the mode controller 140 changes the level of the automatic travel mode AM according to the identification result of the operator OP by the identification function. Therefore, since the automatic travel mode AM suitable for the operator OP can be executed, it is possible to realize a safer automatic travel mode.

The identification function is not limited to the fingerprint authentication function 136. A function for authenticating the finger vein pattern may be used. The level of the automatic travel mode AM is not limited to the level of the travel speed. The mobile radiographic imaging apparatus 2 is made to have a function of detecting an obstacle ahead and prompting an avoidance action in a case where the distance from the obstacle approaches the set distance. Then, the set distance for prompting the avoidance action is changed according to the identification result of the operator OP. More specifically, the set distance increases as the proficiency level decreases, such as the set distance D1 in a case where the proficiency is A, the set distance D2 (>D1) in a case where the proficiency is B, and the set distance D3 (>D2>D1) in a case where the proficiency is C. According to the identification result of the operator OP, whether or not the automatic travel mode AM can be executed may be changed.

### Fourth Embodiment

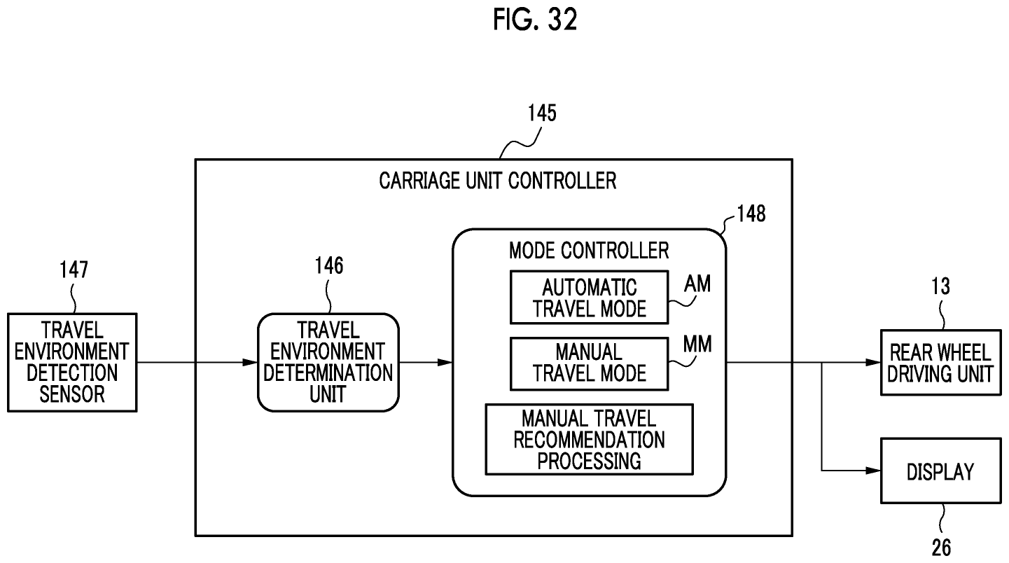

In a fourth embodiment shown in FIGS. 32 to 38, in a case where the travel environment of the carriage unit 10 does not satisfy the recommendation conditions of the automatic travel mode AM during the execution of the automatic travel mode AM, manual travel recommendation processing for recommending switching to the manual travel mode MM is executed.

In FIG. 32, a carriage unit controller 145 of the fourth embodiment has a travel environment determination unit 146 in addition to the route creation unit 90, the first acquisition unit 91, and the second acquisition unit 92 shown in FIG. 8. In the fourth embodiment, a travel environment detection sensor 147 that detects the travel environment of the carriage unit 10 is provided.

The travel environment detection sensor 147 is, for example, a camera that captures a situation in front of the carriage unit 10 during the execution of the automatic travel mode AM. The travel environment detection sensor 147 outputs the captured image to the travel environment determination unit 146. The travel environment determination unit 146 determines whether or not the travel environment of the carriage unit 10 satisfies the recommendation conditions of the automatic travel mode AM based on the image from the travel environment detection sensor 147.

The travel environment determination unit 146 performs image recognition of the image from the travel environment detection sensor 147, and extracts obstacles including a person from the image. Then, the number of times of reflection of the obstacle within the set time is counted. In a case where the counted number of times exceeds the set value, the travel environment determination unit 146 determines that the travel environment of the carriage unit 10 does not satisfy the recommendation conditions of the automatic travel mode AM. In a case where it is determined that the travel environment of the carriage unit 10 does not satisfy the recommendation conditions of the automatic travel mode AM, the travel environment determination unit 146 outputs the determination result to a mode controller 148.

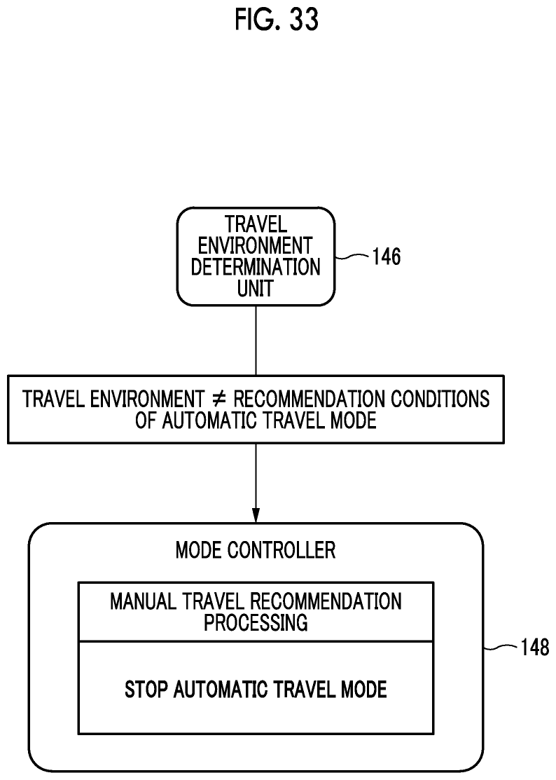

The mode controller 148 receives the determination result, which indicates that the travel environment of the carriage unit 10 does not satisfy the recommendation conditions of the automatic travel mode AM, from the travel environment determination unit 146. In this case, the mode controller 148 executes manual travel recommendation processing for recommending switching to the manual travel mode MM. Specifically, the manual travel recommendation processing is at least one of the modes shown in FIGS. 33 to 35.

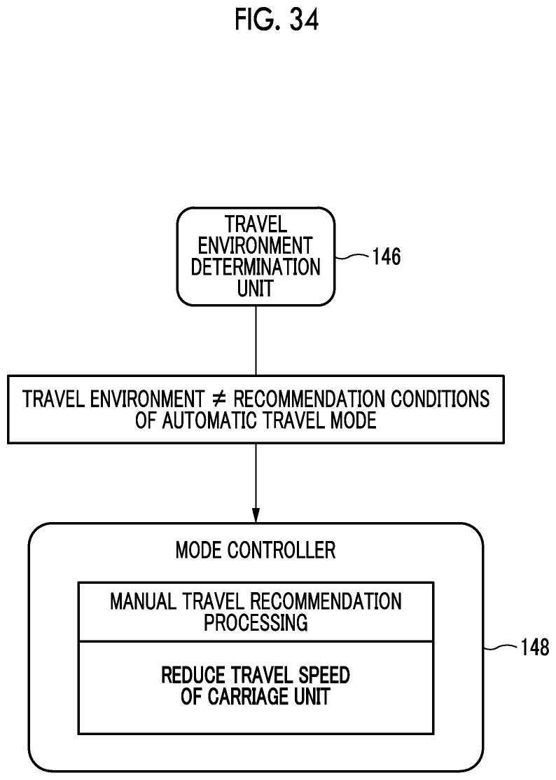

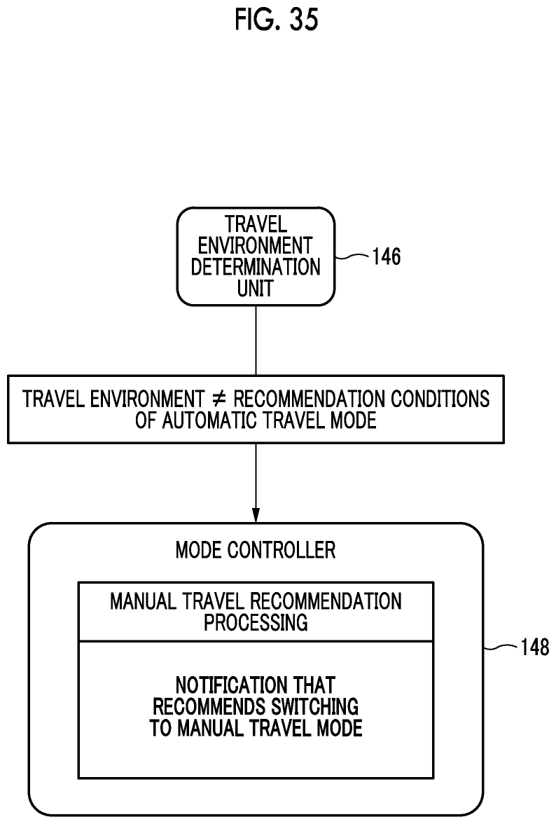

The manual travel recommendation processing shown in FIG. 33 is processing for stopping the automatic travel mode AM by stopping the driving of the rear wheel driving unit 13. The manual travel recommendation processing shown in FIG. 34 is processing for reducing the travel speed of the carriage unit 10 by controlling the rear wheel driving unit 13. The manual travel recommendation processing shown in FIG. 35 is processing for providing notification that recommends switching to the manual travel mode MM through the display 26.

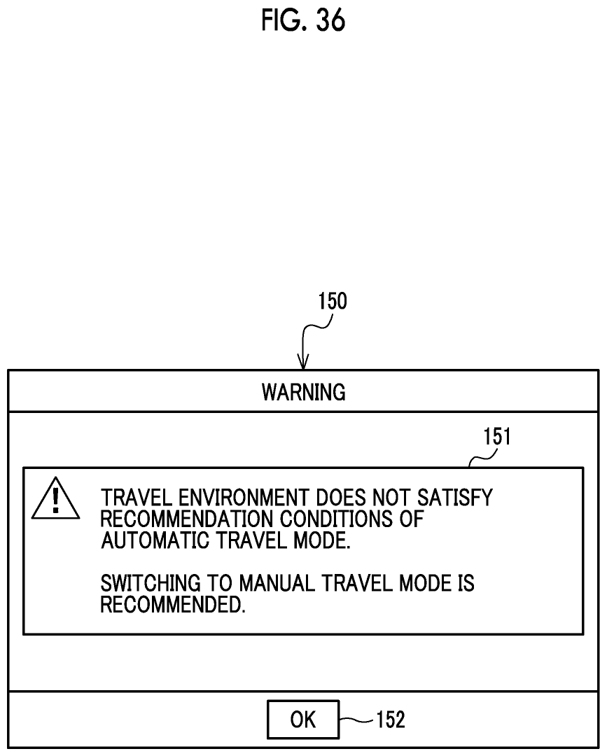

FIG. 36 shows a warning screen 150 displayed on the display 26 in the case of the manual travel recommendation processing shown in FIG. 35. On the warning screen 150, a message 151 and an OK button 152 are displayed. The message 151 is a text indicating that the travel environment of the carriage unit 10 does not satisfy the recommendation conditions for the automatic travel mode AM and that switching to the manual travel mode MM is recommended. The OK button 152 is a button for deleting the display of the warning screen 150.

As described above, in the fourth embodiment, in a case where the travel environment of the carriage unit 10 does not satisfy the recommendation conditions of the automatic travel mode AM during the execution of the automatic travel mode AM, the mode controller 148 executes manual travel recommendation processing for recommending switching to the manual travel mode MM. Therefore, it is possible to ensure the traveling safety of the carriage unit 10.

As shown in FIG. 33, in a case where the manual travel recommendation processing is processing for stopping the automatic travel mode AM, the traveling safety of the carriage unit 10 can be further increased. As shown in FIG. 34, in a case where the manual travel recommendation processing is processing for reducing the travel speed of the carriage unit 10, it is possible to suspend the determination of the operator OP regarding whether or not the switching to the manual travel mode MM is required while continuing the execution of the automatic travel mode AM.

As shown in FIGS. 35 and 36, in a case where the manual travel recommendation processing is processing for providing notification that recommends switching to the manual travel mode MM, the travel environment of the carriage unit 10 does not satisfy the recommendation conditions of the automatic travel mode AM. Therefore, the operator OP can know that it is necessary to switch to the manual travel mode MM.

As described above, the manual travel recommendation processing may be at least one of the modes shown in FIGS. 33 to 35. For this reason, the automatic travel mode AM may be stopped, and the warning screen 150 may be displayed on the display 26. Alternatively, the automatic travel mode AM may be stopped after reducing the travel speed of the carriage unit 10, and the warning screen 150 may be displayed on the display 26. Thus, in a case where the mode for providing notification that recommends switching to the manual travel mode MM is executed in combination with other modes, the operator OP can know the reason why the automatic travel mode AM has been stopped or the reason why the travel speed of the carriage unit 10 has been reduced.

The notification that recommends switching to the manual travel mode MM is not limited to the warning screen 150. Instead of or in addition to displaying the warning screen 150, notification using voice may be provided. Alternatively, a display lamp may be turned on or blinked.

The travel environment detection sensor 147 may be any sensor capable of detecting an obstacle ahead, and is not limited to the above-described camera.

As the travel environment detection sensor 147, a sensor that detects the distance to the obstacle may be used. Then, in a case where the distance to the obstacle is equal to or less than the set value, the travel environment determination unit 146 may determine that the travel environment of the carriage unit 10 does not satisfy the recommendation conditions of the automatic travel mode AM.

Alternatively, a sensor that detects at least one of the inclination or the unevenness of the traveling surface of the carriage unit 10, for example, a micro electromechanical system (MEMS) type motion sensor may be used as the travel environment detection sensor 147. Then, in at least one of a case where the downward inclination of the traveling surface of the carriage unit 10 becomes equal to or greater than the set value or a case where a period for which the unevenness of the traveling surface of the carriage unit 10 is equal to or greater than the set value continues for the set period or longer, the travel environment determination unit 146 may determine that the travel environment of the carriage unit 10 does not satisfy the recommendation conditions of the automatic travel mode AM.

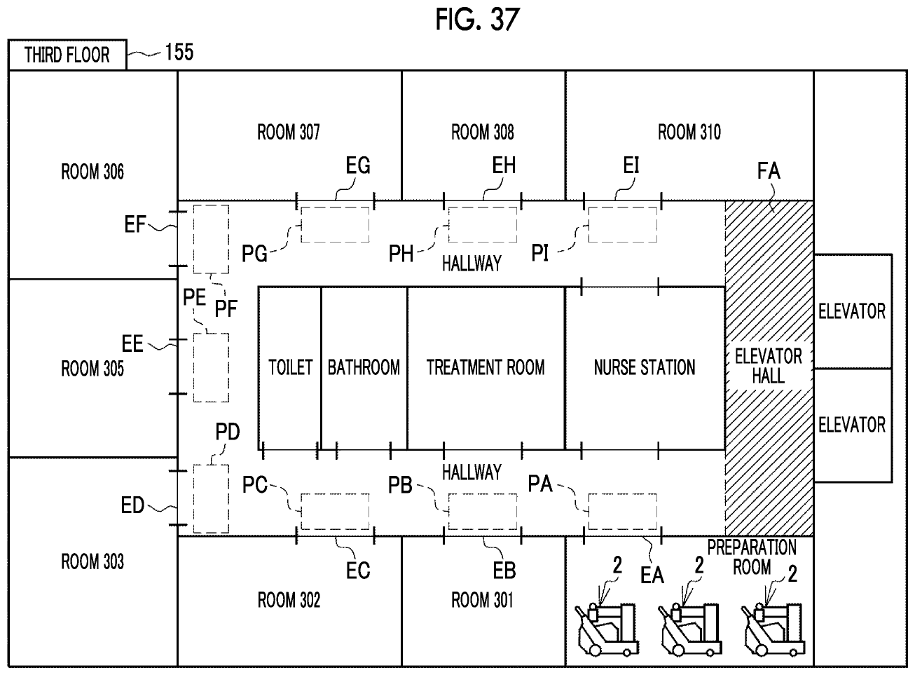

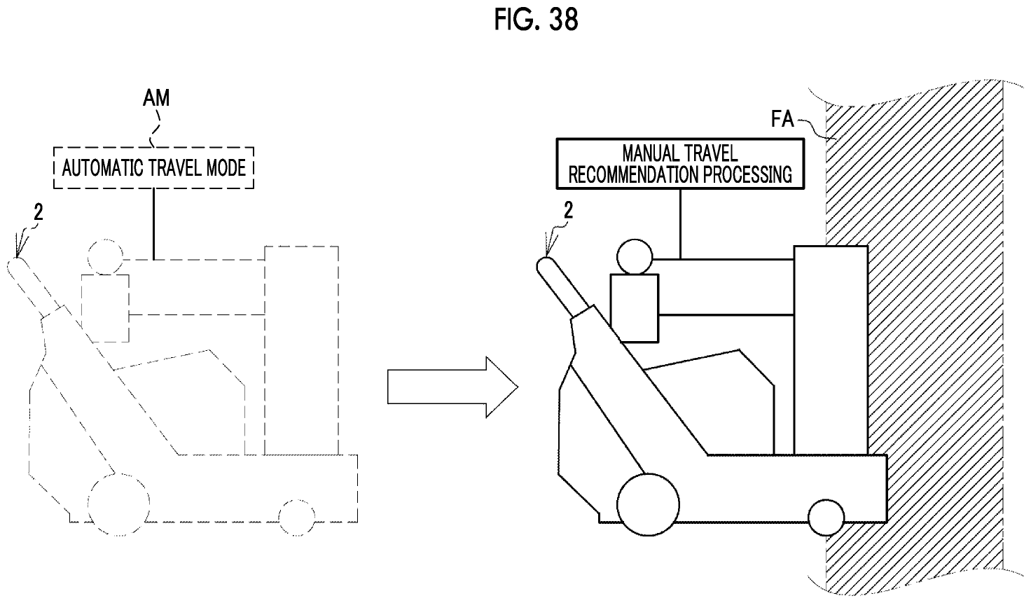

As in map information 155 shown in FIG. 37, a part of the floor may be set in advance as a prohibited area FA of the automatic travel mode AM like the. As shown in FIG. 38, in a case where the carriage unit 10 moves into the prohibited area FA during the execution of the automatic travel mode AM, the travel environment determination unit 146 may determine that the travel environment of the carriage unit 10 does not satisfy the recommendation conditions of the automatic travel mode AM, and the mode controller 148 may execute the manual travel recommendation processing. FIG. 37 shows an example in which an elevator hall is set in the prohibited area of the automatic travel mode AM.

The first acquisition unit 91 and the second acquisition unit 92 may be one acquisition unit.

In each of the embodiments described above, for example, various processors shown below can be used as the hardware structures of processing units that execute various kinds of processing, such as the route creation unit 90, the first acquisition unit 91, the second acquisition unit 92, the mode controllers 93, 140, and 148, and the travel environment determination unit 146. The various processors include not only the above-described CPU, which is a general-purpose processor that executes software (operation program 80) to function as various processing units, but also a programmable logic device (PLD) that is a processor whose circuit configuration can be changed after manufacture, such as a field programmable gate array (FPGA), and/or a dedicated electric circuit that is a processor having a circuit configuration that is designed for exclusive use in order to execute specific processing, such as an application specific integrated circuit (ASIC).

One processing unit may be configured by one of these various processors, or may be a combination of two or more processors of the same type or different types (for example, a combination of a plurality of FPGAs and/or a combination of a CPU and an FPGA). Alternatively, a plurality of processing units may be configured by one processor.

As an example of configuring a plurality of processing units using one processor, first, as represented by a computer, such as a client and a server, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more of the above-described various processors as a hardware structure.

More specifically, as the hardware structure of these various processors, it is possible to use an electrical circuit (circuitry) in which circuit elements, such as semiconductor elements, are combined.

From the above description, it is possible to grasp the invention described in the following supplementary item 1.

Supplementary Item 1

A mobile radiographic imaging apparatus comprising a carriage unit which has wheels for traveling and on which a main body unit is mounted and a handle that is provided in the main body unit to steer the carriage unit, the apparatus comprising: a first acquisition processor that acquires a detection result of a first operation of an operator on the handle from a first detection sensor; a second acquisition processor that acquires a detection result of a second operation of the operator, which is different from the first operation and is performed within a set range that is set in advance around the carriage unit, from a second detection sensor; and a mode control processor that executes or stops an automatic travel mode, in which the carriage unit travels without an operation of the operator on the handle, or a manual travel mode, in which the carriage unit travels by an operation of the operator on the handle, according to a detection result of the first operation and a detection result of the second operation and that continues execution of the automatic travel mode in a case where the first operation is not detected by the first detection sensor and the second operation is continuously detected by the second detection sensor and stops the automatic travel mode and executes the manual travel mode in a case where the first operation is detected by the first detection sensor during execution of the automatic travel mode.

According to the technique of the present disclosure, it is possible to appropriately combine at least any two of the above-described various embodiments and various modification examples. In addition, it is needless to say that, without being limited to the embodiments described above, various configurations can be adopted without departing from the scope of the present disclosure. In addition, the technique of the present disclosure extends not only to a program but also to a storage medium that stores a program non-temporarily.

The described content and the illustrated content above are detailed descriptions of portions relevant to the technique of the present disclosure, and are merely examples of the technique of the present disclosure. For example, the above descriptions regarding the configurations, functions, operations, and effects are descriptions regarding examples of the configurations, functions, operations, and effects of portions relevant to the technique of the present disclosure. Therefore, it is needless to say that unnecessary portions may be deleted, new elements may be added, or replacement may be performed for the described content and the illustrated content above without departing from the spirit of the technique of the present disclosure. In addition, in order to avoid complications and facilitate understanding of the portions relevant to the technique of the present disclosure, descriptions regarding common technical knowledge and the like for which descriptions for enabling the implementation of the technique of the present disclosure are not required in particular are omitted.

In this specification, “A and/or B” is synonymous with “at least one of A or B”. That is, “A and/or B” may be only A, only B, or a combination of A and B. In addition, in this specification, the same concept as “A and/or B” is applied to a case where three or more things are expressed with “and/or”.

All documents, patent applications, and technical standards described in this specification are incorporated in this specification by reference to the same extent as in a case where the incorporation of individual documents, patent applications, and technical standards by reference is described specifically and individually.

What is claimed is:

1. A mobile radiographic imaging apparatus, comprising:
a carriage unit which has wheels for traveling and on which a main body unit is mounted;
a handle that is provided in the main body unit to steer the carriage unit;
a first detection sensor that detects a first operation of an operator on the handle;
a second detection sensor that detects a second operation of the operator, which is different from the first operation and is performed within a set range that is set in advance around the carriage unit; and
a mode controller that executes or stops an automatic travel mode, in which the carriage unit travels without an operation of the operator on the handle, or a manual travel mode, in which the carriage unit travels by an operation of the operator on the handle, according to a detection result of the first operation and a detection result of the second operation and that continues execution of the automatic travel mode in a case where the first operation is not detected by the first detection sensor and the second operation is continuously detected by the second detection sensor and stops the automatic travel mode and executes the manual travel mode in a case where the first operation is detected by the first detection sensor during execution of the automatic travel mode.

2. The mobile radiographic imaging apparatus according to claim 1,
wherein, in a case where the second operation is not detected by the second detection sensor, the mode controller stops the automatic travel mode.

3. The mobile radiographic imaging apparatus according to claim 1,
wherein the first detection sensor detects that the operator has gripped the handle as the first operation, and
the second detection sensor is provided in the main body unit, and detects that one hand of the operator has come into contact with the second detection sensor itself as the second operation.

4. The mobile radiographic imaging apparatus according to claim 3,
wherein the second detection sensor is attached to one end of an extension cable, and is usable by being detached from the main body unit.

5. The mobile radiographic imaging apparatus according to claim 3,
wherein the second detection sensor has a wireless transmission function for wirelessly transmitting the detection result of the second operation within a communication range limited by the set range.

6. The mobile radiographic imaging apparatus according to claim 3,
wherein the first detection sensor is at least one of a sensor that detects contact of a hand of the operator with the handle, a camera that images a gripping state of the handle by the operator, or a sensor that detects that a force for steering the carriage unit has been applied to the handle.

7. The mobile radiographic imaging apparatus according to claim 1,
wherein the first detection sensor detects that the operator has gripped the handle with both hands as the first operation, and
the second detection sensor detects that one hand of the operator has come into contact with the handle as the second operation.

8. The mobile radiographic imaging apparatus according to claim 7,
wherein each of the first detection sensor and the second detection sensor is at least one of a sensor that detects contact of a hand of the operator with the handle or a camera that images a gripping state of the handle by the operator.

9. The mobile radiographic imaging apparatus according to claim 1,
wherein the second detection sensor has an identification function for identifying the operator.

10. The mobile radiographic imaging apparatus according to claim 9,
wherein the mode controller changes a level of the automatic travel mode according to an identification result of the operator by the identification function.

11. The mobile radiographic imaging apparatus according to claim 1,
wherein, in a case where a travel environment of the carriage unit does not satisfy recommendation conditions of the automatic travel mode during execution of the automatic travel mode, the mode controller executes manual travel recommendation processing for recommending switching to the manual travel mode.

12. The mobile radiographic imaging apparatus according to claim 11,
wherein the manual travel recommendation processing is processing for stopping the automatic travel mode.

13. The mobile radiographic imaging apparatus according to claim 11,
wherein the manual travel recommendation processing is processing for reducing a travel speed of the carriage unit.

14. The mobile radiographic imaging apparatus according to claim 11,
wherein the manual travel recommendation processing is processing for providing notification that recommends switching to the manual travel mode.

15. The mobile radiographic imaging apparatus according to claim 1,
wherein a parking position of the carriage unit in the automatic travel mode is a position corresponding to a schedule of radiographic imaging, and includes a position outside a patient room that is defined in advance for each patient room.

16. An operation method of a mobile radiographic imaging apparatus comprising a carriage unit which has wheels for traveling and on which a main body unit is mounted and a handle that is provided in the main body unit to steer the carriage unit, the method comprising:
a first acquisition step of acquiring a detection result of a first operation of an operator on the handle from a first detection sensor;
a second acquisition step of acquiring a detection result of a second operation of the operator, which is different from the first operation and is performed within a set range that is set in advance around the carriage unit, from a second detection sensor; and
a mode control step of executing or stopping an automatic travel mode, in which the carriage unit travels without an operation of the operator on the handle, or a manual travel mode, in which the carriage unit travels by an operation of the operator on the handle, according to a detection result of the first operation and a detection result of the second operation and of continuing execution of the automatic travel mode in a case where the first operation is not detected by the first detection sensor and the second operation is continuously detected by the second detection sensor and stopping the automatic travel mode and executing the manual travel mode in a case where the first operation is detected by the first detection sensor during execution of the automatic travel mode.

17. A non-transitory computer-readable storage medium storing an operation program of a mobile radiographic imaging apparatus comprising a carriage unit which has wheels for traveling and on which a main body unit is mounted and a handle that is provided in the main body unit to steer the carriage unit, the operation program causing a computer to function as:
a first acquisition unit that acquires a detection result of a first operation of an operator on the handle from a first detection sensor;
a second acquisition unit that acquires a detection result of a second operation of the operator, which is different from the first operation and is performed within a set range that is set in advance around the carriage unit, from a second detection sensor; and
a mode controller that executes or stops an automatic travel mode, in which the carriage unit travels without an operation of the operator on the handle, or a manual travel mode, in which the carriage unit travels by an operation of the operator on the handle, according to a detection result of the first operation and a detection result of the second operation and that continues execution of the automatic travel mode in a case where the first operation is not detected by the first detection sensor and the second operation is continuously detected by the second detection sensor and stops the automatic travel mode and executes the manual travel mode in a case where the first operation is detected by the first detection sensor during execution of the automatic travel mode.
